(19) United States
(12) Patent Application Publication
(10) Pub. No.: US 2006/0257450 A1
(43) Pub. Date: Nov. 16, 2006

(54) Drug delivery systems for treatment of diseases or conditions

(76) Inventors: Sreenivasu Mudumba, Union City, CA (US); Philippe Jm Dor, Cupertino, CA (US); Thierry Nivaggioli, Atherton, CA (US); David A. Weber, Danville, CA (US); Sidiq Farooq, Newark, CA (US)

Correspondence Address: MORRISON & FOERSTER LLP, 755 PAGE MILL RD, PALO ALTO, CA, 94304-1018, US

(21) Appl. No.: 11/386,290

(22) Filed: Mar. 21, 2006

Related U.S. Application Data
(60) Provisional application No. 60/664,119, filed on Mar. 21, 2005.
(60) Provisional application No. 60/666,872, filed on Mar. 30, 2005.

(51) Int. Cl. A61K 31/4745; A61F 2/02

(52) U.S. Cl. 424/427; 514/291

(57) ABSTRACT

Diseases and conditions associated with tissues of the body, including but not limited to tissues in the eye, can be effectively treated, prevented, inhibited, onset delayed, or regression caused by administering therapeutic agents to those tissues. Described herein are solid drug delivery systems and methods for providing extended delivery of therapeutic agents to such tissues. A solid drug delivery system may be placed in a subject, including but not limited to placement between the sclera and the conjunctiva or transscleral placement. Described methods may be used to administer rapamycin to treat or prevent angiogenesis, choroidal neovascularization, age-related macular degeneration, or wet age-related macular degeneration in a subject. The solid drug delivery devices may comprise rapamycin or other therapeutic agents. Also described are methods of treating ocular diseases or disorders by administering an antiproliferative agent, including but not limited to rapamycin, proximal to an ocular device.

### CROSS-REFERENCE TO RELATED APPLICATIONS

The present application is related to and claims priority from U.S. Provisional Patent Application Ser. No. 60/664,119 titled “DRUG DELIVERY SYSTEMS FOR TREATMENT OF DISEASES OR CONDITIONS,” filed Mar. 21, 2005, and U.S. Provisional Patent Application Ser. No. 60/666,872 titled “GLAUCOMA DRAINAGE DEVICES,” filed Mar. 30, 2005, each of which is incorporated herein by reference in its entirety for all purposes.

### FIELD

Described herein are solid drug delivery systems to treat, prevent, inhibit, delay onset of, or cause regression of a disease or condition by delivery of therapeutic agents to a subject, including but not limited to a human subject, including but not limited to the treatment of age-related macular degeneration (“AMD”) by placement of a solid drug delivery system comprising rapamycin (sirolimus), to the eye of a subject.

### BACKGROUND

The retina of the eye contains the cones and rods that detect light. In the center of the retina is the macula lutea, which is about ⅓ to ½ cm in diameter. The macula provides detailed vision, particularly in the center (the fovea), because the cones are higher in density. Blood vessels, ganglion cells, inner nuclear layer and cells, and the plexiform layers are all displaced to one side (rather than resting above the cones), thereby allowing light a more direct path to the cones.

Under the retina are the choroid, comprising a collection of blood vessels embedded within a fibrous tissue, and the deeply pigmented epithelium, which overlays the choroid layer. The choroidal blood vessels provide nutrition to the retina (particularly its visual cells).

There are a variety of retinal disorders for which there is currently no treatment or for which the current treatment is not optimal. Retinal disorders such as uveitis (an inflammation of the uveal tract: iris, ciliary body, and choroid), central retinal vein occlusive diseases (CRVO), branch retinal venous occlusion (BRVO), macular degeneration, macular edema, proliferative diabetic retinopathy, and retinal detachment generally are all retinal disorders that are difficult to treat with conventional therapies.

Age-related macular degeneration (AMD) is the major cause of severe visual loss in the United States for individuals over the age of 60. AMD occurs in either an atrophic or less commonly an exudative form. The atrophic form of AMD is also called “dry AMD,” and the exudative form of AMD is also called “wet AMD.”

In exudative AMD, blood vessels grow from the choriocapillaris through defects in Bruch's membrane, and in some cases the underlying retinal pigment epithelium. Organization of serous or hemorrhagic exudates escaping from these vessels results in fibrovascular scarring of the macular region with attendant degeneration of the neuroretina, detachment and tears of the retinal pigment epithelium, vitreous hemorrhage and permanent loss of central vision. This process is responsible for more than 80% of cases of significant visual loss in subjects with AMD. Current or forthcoming treatments include laser photocoagulation, photodynamic therapy, treatment with VEGF antibody fragments, treatment with pegylated aptamers, and treatment with certain small molecule agents.

Several studies have recently described the use of laser photocoagulation in the treatment of initial or recurrent neovascular lesions associated with AMD (Macular Photocoagulation Study Groups (1991) in Arch. Ophthal. 109:1220; Arch. Ophthal. 109:1232; Arch. Ophthal. 109:1242). Unfortunately, AMD subjects with subfoveal lesions subjected to laser treatment experienced a rather precipitous reduction in visual acuity (mean 3 lines) at 3 months follow-up. Moreover, at two years post-treatment treated eyes had only marginally better visual acuity than their untreated counterparts (means of 20/320 and 20/400, respectively). Another drawback of the procedure is that vision after surgery is immediately worse.

Photodynamic therapy (PDT) is a form of phototherapy, a term encompassing all treatments that use light to produce a beneficial reaction in a subject. Optimally, PDT destroys unwanted tissue while sparing normal tissue. Typically, a compound called a photosensitizer is administered to the subject. Usually, the photosensitizer alone has little or no effect on the subject. When light, often from a laser, is directed onto a tissue containing the photosensitizer, the photosensitizer is activated and begins destroying targeted tissue. Because the light provided to the subject is confined to a particularly targeted area, PDT can be used to selectively target abnormal tissue, thus sparing surrounding healthy tissue. PDT is currently used to treat retinal diseases such as AMD. PDT is currently the mainstay of treatment for subfoveal choroidal neovascularization in subjects with AMD (Photodynamic Therapy for Subfoveal Choroidal Neovascularization in Age Related Macular Degeneration with Verteporfin (TAP Study Group) Arch Ophthalmol. 1999 117:1329-1345.

Choroidal neovascularization (CNV) has proven to be recalcitrant to treatment in most cases. Conventional laser treatment can ablate CNV and help to preserve vision in selected cases not involving the center of the retina, but this is limited to only about 10% of the cases. Unfortunately, even with successful conventional laser photocoagulation, the neovascularization recurs in about 50-70% of eyes (50% over 3 years and >60% at 5 years). (Macular Photocoagulation Study Group, Arch. Ophthalmol. 204:694-701 (1986)). In addition, many subjects who develop CNV are not good candidates for laser therapy because the CNV is too large for laser treatment, or the location cannot be determined so that the physician cannot accurately aim the laser. Photodynamic therapy, although utilized in up to 50% of new cases of subfoveal CNV has only marginal benefits over natural history, and generally delays progression of visual loss rather than improving vision which is already decreased secondary to the subfoveal lesion. PDT is neither preventive or definitive. Several PDT treatments are usually required per subject and additionally, certain subtypes of CNV fare less well than others.

Thus, there remains a long-felt need for methods and solid drug delivery systems that may be used to optimally prevent or significantly inhibit choroidal neovascularization and to prevent and treat wet AMD.

In addition to AMD, choroidal neovascularization is associated with such retinal disorders as presumed ocular histoplasmosis syndrome, myopic degeneration, angioid streaks, idiopathic central serous chorioretinopathy, inflammatory conditions of the retina and or choroid, and ocular trauma. Angiogenic damage associated with neovascularization occurs in a wide range of disorders including diabetic retinopathy, venous occlusions, sickle cell retinopathy, retinopathy of prematurity, retinal detachment, ocular ischemia and trauma.

Uveitis is another retinal disorder that has proven difficult to treat using existing therapies. Uveitis is a general term that indicates an inflammation of any component of the uveal tract. The uveal tract of the eye consists of the iris, ciliary body, and choroid. Inflammation of the overlying retina, called retinitis, or of the optic nerve, called optic neuritis, may occur with or without accompanying uveitis.

Uveitis is most commonly classified anatomically as anterior, intermediate, posterior, or diffuse. Posterior uveitis signifies any of a number of forms of retinitis, choroiditis, or optic neuritis. Diffuse uveitis implies inflammation involving all parts of the eye, including anterior, intermediate, and posterior structures.

The symptoms and signs of uveitis may be subtle, and vary considerably depending on the site and severity of the inflammation. Regarding posterior uveitis, the most common symptoms include the presence of floaters and decreased vision. Cells in the vitreous humor, white or yellow-white lesions in the retina and/or underlying choroid, exudative retinal detachments, retinal vasculitis, and optic nerve edema may also be present in a subject suffering from posterior uveitis.

Ocular complications of uveitis may produce profound and irreversible loss of vision, especially when unrecognized or treated improperly. The most frequent complications of posterior uveitis include retinal detachment; neovascularization of the retina, optic nerve, or iris; and cystoid macular edema.

Macular edema (ME) can occur if the swelling, leaking, and hard exudates noted in background diabetic retinopathy (BDR) occur within the macula, the central 5% of the retina most critical to vision. Background diabetic retinopathy (BDR) typically consists of retinal microaneurisms that result from changes in the retinal microcirculation. These microaneurisms are usually the earliest visible change in retinopathy seen on exam with an ophthalmoscope as scattered red spots in the retina where tiny, weakened blood vessels have ballooned out. The ocular findings in background diabetic retinopathy progress to cotton wool spots, intraretinal hemorrhages, leakage of fluid from the retinal capillaries, and retinal exudates. The increased vascular permeability is also related to elevated levels of local growth factors such as vascular endothelial growth factor. The macula is rich in cones, the nerve endings that detect color and upon which daytime vision depends. When increased retinal capillary permeability effects the macula, blurring occurs in the middle or just to the side of the central visual field, rather like looking through cellophane. Visual loss may progress over a period of months, and can be very annoying because of the inability to focus clearly. ME is a common cause of severe visual impairment.

There have been many attempts to treat CNV and its related diseases and conditions, as well as other conditions such as macular edema and chronic inflammation, with pharmaceuticals. For example, use of rapamycin to inhibit CNV and wet AMD has been described in U.S. application Ser. No. 10/665,203, which is incorporated herein by reference in its entirety. The use of rapamycin to treat inflammatory diseases of the eye has been described in U.S. Pat. No. 5,387,589, titled Method of Treating Ocular Inflammation, with inventor Prassad Kulkami, assigned to University of Louisville Research Foundation, the contents of which is incorporated herein in its entirety.

Particularly for chronic diseases, including those described herein, there is a great need for long acting methods for delivering therapeutic agents to the eye, such as to the posterior segment to treat CNV in such diseases as AMD, macular edema, proliferative retinopathies, and chronic inflammation. Delivery systems with extended delivery of therapeutic agent are more comfortable and convenient for a subject, due to a diminished frequency of ocular placement of the solid drug delivery system.

Direct delivery of therapeutic agents to the eye rather than systemic administration may be advantageous because the therapeutic agent concentration at the site of action is increased relative to the therapeutic agent concentration in a subject's circulatory system. Additionally, therapeutic agents may have undesirable side effects when delivered systemically to treat posterior segment disease. Thus, localized drug delivery may promote efficacy while decreasing side effects and systemic toxicity.

### SUMMARY

The methods and solid drug delivery systems described herein allow delivery of a therapeutic agent to a subject, including but not limited to a human subject or to the eye of a subject. Described herein are methods and solid drug delivery systems for delivering a variety of therapeutic agents for the treatment, prevention, inhibition, delaying onset of, or causing regression of a number of conditions or diseases, including but not limited to diseases or conditions of the eye. Also described herein are methods of administering an antiproliferative agent proximal to an ocular device to treat a disease or condition of the eye; in some variations the ocular device is a glaucoma drainage device.

Described herein are solid drug delivery systems comprising rapamycin, and wherein the solid drug delivery system when placed between the sclera and conjunctiva of a rabbit eye delivers an amount of rapamycin with a delivery profile selected from the group consisting of (a) the rapamycin is delivered in an amount sufficient to achieve, for a period of time of at least 90 days following administration of the solid drug delivery system, an average concentration of rapamycin in the vitreous of the rabbit eye of at least 0.01 ng/ml; and (b) the rapamycin is delivered in an amount sufficient to achieve, for a period of time of at least 90 days following administration of the solid drug delivery system, an average concentration of rapamycin in the retina choroid of the rabbit eye of at least 1 pg/mg.

In some variations the solid drug delivery system when placed between the sclera and conjunctiva of a rabbit eye delivers an amount of rapamycin with a delivery profile selected from the group consisting of (a) the rapamycin is delivered in an amount sufficient to achieve, for a period of time of at least 90 days following administration of the solid drug delivery system, an average concentration of rapamycin in the vitreous of the rabbit eye of at least 0.1 ng/ml; and (b) the rapamycin is delivered in an amount sufficient to achieve, for a period of time of at least 90 days following administration of the solid drug delivery system, an average concentration of rapamycin in the retina choroid of the rabbit eye of at least 10 pg/mg.

In some variations the solid drug delivery system when placed between the sclera and conjunctiva of a rabbit eye delivers an amount of rapamycin with a delivery profile selected from the group consisting of (a) the rapamycin is delivered in an amount sufficient to achieve, for a period of time of at least 90 days following administration of the solid drug delivery system, an average concentration of rapamycin in the vitreous of the rabbit eye of at least 1 ng/ml; and (b) the rapamycin is delivered in an amount sufficient to achieve, for a period of time of at least 90 days following administration of the solid drug delivery system, an average concentration of rapamycin in the retina choroid of the rabbit eye of at least 100 pg/mg.

Described herein are solid drug delivery systems comprising a therapeutic agent, wherein the solid drug delivery system when placed between the sclera and conjunctiva of a rabbit eye delivers an amount of the therapeutic agent with a delivery profile selected from the group consisting of (a) the the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.01 ng/ml; and (b) the the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 1 pg/mg. In some variations the therapeutic agent is a limus compound. In some variations the therapeutic agent is selected from the group consisting of rapamycin, SDZ-RAD, tacrolimus, everolimus, pimecrolimus, CCI-779, AP23841, ABT-578, cyclophilins, TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, and pharmaceutically acceptable salts and esters thereof. In some variations the therapeutic agent is selected from the group consisting of rapamycin, SDZ-RAD, tacrolimus, everolimus, pimecrolimus, CCI-779, AP23841, ABT-578, and pharmaceutically acceptable salts and esters thereof.

Described herein are solid drug delivery systems comprising a backing portion that is at least partially impermeable to the therapeutic agent.

Described herein are solid drug delivery systems, wherein the solid drug delivery system when placed between the sclera and conjunctiva of a rabbit eye delivers an amount of the therapeutic agent with a delivery profile selected from the group consisting of (a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.1 ng/ml; and (b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 10 pg/mg. In some variations the solid drug delivery system when placed between the sclera and conjunctiva of a rabbit eye delivers an amount of the therapeutic agent with a delivery profile selected from the group consisting of (a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 1 ng/ml; and (b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 0.05 pg/mg.

Described herein are solid drug delivery systems comprising a therapeutic agent, including but not limited to rapamycin, in an amount between 1% and 60% w/w of the drug delivery system.

Described herein are solid drug delivery systems comprising a polyvinylpyrrolidone in an amount between 15% and 45% w/w of the solid drug delivery system.

Described herein are solid drug delivery systems comprising a polyacrylate in an amount between 5% and 30% w/w of the solid drug delivery system.

Described herein are solid drug delivery systems wherein the therapeutic agent, including but not limited to rapamycin, is present in an amount between 1% and 60% w/w of the drug delivery system, further comprising a polyvinylpyrrolidone present in an amount between 15% and 45% w/w of the solid drug delivery system, and a polyacrylate present in an amount between 5% and 30% w/w of the solid drug delivery system.

In some variations, the solid drug delivery system contains between 20 μg and 4 mg of rapamycin. In some variations, the solid drug delivery system contains between 20 μg and 2.5 mg of rapamycin.

Described herein are methods for treating wet age-related macular degeneration in a human subject, the method comprising placing a solid drug delivery system described herein proximal to the eye of a human subject in need of treatment of age related macular degeneration.

Described herein are methods for preventing wet age-related macular degeneration in a human subject, the method comprising placing a solid drug delivery system described herein proximal to the eye of the human subject in need of prevention of age related macular degeneration. In some variations the human subject is identified as being at heightened risk of developing wet age-related macular degeneration in the eye to which the solid drug delivery system is administered. In some variations the human subject has dry age-related macular degeneration in at least one eye. In some variations the human subject has wet age-related macular degeneration in one eye and the solid drug delivery system is administered to the eye without wet age-related macular degeneration.

In some variations, the eye has a sclera with an outer scleral surface and a solid drug delivery system described herein is placed proximal to the outer scleral surface or within a scleral flap. In some variations, a solid drug delivery system described herein is placed between the sclera and conjunctiva.

Described herein are solid drug delivery systems comprising a therapeutic agent, a polyvinylpyrrolidone, and a polyacrylate, wherein the therapeutic agent is selected from the group consisting of rapamycin, SDZ-RAD, tacrolimus, everolimus, pimecrolimus, CCI-779, AP23841, ABT-578, cyclophilins, TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, and pharmaceutically acceptable salts and esters thereof.

Described herein are solid drug delivery systems comprising a limus compound, a polyvinylpyrrolidone, and a polyacrylate.

In some variations the therapeutic agent is selected from the group consisting of rapamycin, SDZ-RAD, tacrolimus, everolimus, pimecrolimus, CCI-779, AP23841, ABT-578, and pharmaceutically acceptable salts and esters thereof.

In some variations the solid drug delivery systems described herein further comprise a polyethylene glycol.

In some variations the solid drug delivery system when placed between the sclera and conjunctiva of a rabbit eye delivers an amount of the therapeutic agent with a delivery profile selected from the group consisting of (a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.1 ng/ml; and (b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 0.01 ng/mg.

In some variations the solid drug delivery system when placed between the sclera and conjunctiva of a rabbit eye delivers an amount of the therapeutic agent with a delivery profile selected from the group consisting of (a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.5 ng/ml; and (b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 0.05 ng/mg.

In some variations the excipient component comprises a solvent. The solvent may be a liquid or a solid, either prior or subsequent to mixing with the therapeutic agent.

In some variations the excipient component comprises a release modifying agent.

In some variations the excipient component comprises a solubilizing agent.

In one method, a solid drug delivery system comprising a therapeutic agent including but not limited to rapamycin, and an excipient component, is placed in a subject to treat, prevent, inhibit, delay of the onset of, or cause the regression of a disease or condition of the eye.

As described in further detail in the Detailed Description section, the methods and solid drug delivery systems may also be used for delivery to a subject, including but not limited to a human subject or to the eye of a human subject of therapeutically effective amounts of rapamycin for the treatment, prevention, inhibition, delaying of the onset of, or causing the regression of wet AMD. In some variations, the methods and solid drug delivery systems are used to treat wet AMD. In some variations, the methods and solid drug delivery systems are used to prevent wet AMD. In some variations, the methods and solid drug delivery systems described herein are used to prevent the transition from dry AMD to wet AMD. The methods and solid drug delivery systems may also be used for delivery to a subject, including but not limited to a human subject or to the eye of a subject of therapeutically effective amounts of rapamycin for the treatment, prevention, inhibition, delaying of the onset of, or causing the regression of CNV. In some variations, the methods and solid drug delivery systems are used to treat CNV. The methods and solid drug delivery systems may also be used for delivery to a subject, including but not limited to a human subject or to the eye of a subject of therapeutically effective amounts of rapamycin for the treatment, prevention, inhibition, delaying of the onset of, or causing the regression of angiogenesis in the eye. In some variations, the methods and solid drug delivery systems are used to treat angiogenesis. Other diseases and conditions that may be treated, prevented, inhibited, have onset delayed, or caused to regress using rapamycin are described in the

Diseases and Conditions Section of the Detailed Description.

As described in further detail in the Detailed Description, the methods and solid drug delivery systems may also be used for delivery to a subject, including but not limited to a human subject or to the eye of a subject of therapeutically effective amounts of therapeutic agents other than rapamycin for the treatment, prevention, inhibition, delaying of the onset of, or causing the regression of wet AMD. In some variations, the methods and solid drug delivery systems are used to treat wet AMD. Therapeutic agents that may be used are described in detail in the Therapeutic Agents section. Such therapeutic agents include but are not limited to immunophilin binding compounds. Immunophilin binding compounds that may be used include but are not limited to the limus family of compounds described further in the Therapeutic Agents section herein, including rapamycin, SDZ-RAD, tacrolimus, everolimus, pimecrolimus, CCI-779, AP23841, ABT-578, derivatives, analogs, prodrugs, salts and esters thereof. The methods and solid drug delivery systems may also be used for delivery to a subject, including but not limited to a human subject or to the eye of a subject of therapeutically effective amounts of therapeutic agents for the treatment, prevention, inhibition, delaying of the onset of, or causing the regression of CNV. In some variations, the methods and solid drug delivery systems are used to treat CNV. The methods and solid drug delivery systems may also be used for delivery to a subject, including but not limited to a human subject or to the eye of a subject of therapeutically effective amounts of therapeutic agents for the treatment, prevention, inhibition, delaying of the onset of, or causing the regression of angiogenesis in the eye. In some variations, the methods and solid drug delivery systems are used to treat angiogenesis. Other diseases and conditions that may be treated, prevented, inhibited, have onset delayed, or caused to regress using therapeutic agents other than rapamycin are described in the Diseases and Conditions section of the Detailed Description.

The solid drug delivery systems described herein may be biodegradable or non-biodegradable. Placement includes but is not limited to placement of the solid drug delivery system by injection or placement with forceps in a surgical incision, delivery by a polymer-based solid drug delivery system, delivery by a bioadhesive solid drug delivery system, delivery by solid drug delivery system with delayed release, and delivery by a coated solid drug delivery system.

The solid drug delivery system may also optionally include various means for assisting in anchoring the solid drug delivery system in place. As one nonlimiting example, such a solid drug delivery system may include a bioadhesive layer for placement on the outer scleral surface of the eye. In some variations, a solid drug delivery system has a surface containing a number of protrusions which assist in anchoring the solid drug delivery system to the outer scleral surface of the eye. As another nonlimiting example, a solid drug delivery system is sutured to the sclera or other tissue.

The solid drug delivery system may also optionally be a delayed release solid drug delivery system.

The solid drug delivery systems described herein may deliver a therapeutic agent or agents, including but not limited to rapamycin, for an extended period of time. One nonlimiting example of such an extended release delivery system is a solid drug delivery system that delivers a therapeutic agent or agents to a subject or to the eye of a subject in an amount sufficient to maintain an amount effective to treat, prevent, inhibit, delay of the onset of, or cause the regression of a disease or condition in a subject for an extended period of time. In one nonlimiting example, such a delivery system delivers the therapeutic agent for at least about one, about two, about three, about six, about nine, or about twelve months.

Other extended periods of release are described in the Detailed Description.

The solid drug delivery systems described herein may also deliver a therapeutic agent in an amount equivalent to various specified concentrations or levels of rapamycin.

Generally, any concentration of therapeutic agent that has the desired effect can be used. The solid drug delivery system may generally be administered in any amount or size that has the desired effect. The solid drug delivery systems described herein may deliver a therapeutic agent or agents for an extended period of time. One nonlimiting example of such an extended release delivery system is a solid drug delivery system that delivers a therapeutic agent or agents to a subject, including but not limited to a human subject or to the eye of a subject in an amount sufficient to maintain an amount effective to treat, prevent, inhibit, delay onset of, or cause regression of a disease or condition in a subject for an extended period of time. In some variations, the solid drug delivery system is used to treat a disease or condition in a subject, including but not limited to a human subject. In some variations, the solid drug delivery system delivers the therapeutic agent for at least about one, about two, about three, about six, about nine, or about twelve months.

In some variations, the solid drug delivery system is used to prevent wet age-related macular degeneration for an extended period of time. In some variations, the solid drug delivery system is used to prevent transition of dry AMD to wet AMD for an extended period of time. In one nonlimiting example, the solid drug delivery system delivers the rapamycin to the vitreous, sclera, retina, choroid, macula, or other tissues of a subject, including but not limited to a human subject in an amount sufficient to treat, prevent, inhibit, delay onset of, or cause regression of wet age-related macular degeneration for at least about three, about six, about nine, or about twelve months. In some variations, the level of rapamycin is sufficient to treat AMD. In some variations, the level of rapamycin is sufficient to prevent onset of wet AMD. Other extended periods of release are described in the Detailed Description.

Described herein are methods of treating an ocular condition in a subject requiring placement of an ocular device, comprising administering a formulation comprising an anti-proliferative agent proximal to the site selected for placement of the ocular device. In some variations the formulation is administered prior to, contemporaneous with, or subsequent to placement of the ocular device. In some variations the anti-proliferative agent is a limus compound, or a pharmaceutically acceptable salt or ester thereof. In some variations the limus compound is rapamycin. In some variations the ocular device is a glaucoma drainage device. In some variations the ocular device comprises a shunt, stent, tube, membrane, valve, or combination of one or more thereof. In some variations the method reduces cellular proliferation proximal to the ocular device. In some variations the formulation is a solution, suspension, emulsion, self-emulsifying formulation, in situ gelling formulation, or a solid drug delivery system. In some variations the formulation delivers an amount of the antiproliferative agent effective to reduce cellular proliferation proximal to the ocular device for a period of at least about 30 days. In some variations the formulation delivers an amount of the therapeutic agent effective to reduce cellular proliferation proximal to the ocular device for a period of at least about 60 days. In some variations the formulation delivers an amount of the therapeutic agent effective to reduce cellular proliferation proximal to the ocular device for a period of at least about 90 days. In some variations the antiproliferative agent is rapamycin and the ocular device is a glaucoma drainage device.

### BRIEF DESCRIPTION OF THE DRAWINGS

FIG. 1 depicts the level of rapamycin in the vitreous (ng/ml), retina choroid (ng/mg), and sclera (ng/mg) of rabbit eyes at 1, 14, 28, 75, 95 and 107 days after subconjunctival placement of a solid drug delivery system made of 47.7% rapamycin, 23.25% PVP K90, 5.8% PEG 400, and 23.25% Eudragit.

FIG. 2 depicts the level of rapamycin in the retina choroid (ng/mg) of rabbit eyes at 1, 5, 7 and 8 days after subconjunctival placement of a solid drug delivery system made of 10.2% rapamycin and 89.8% PVP K90.

FIG. 3 depicts the level of rapamycin in the vitreous (ng/ml) of rabbit eyes at 1, 5, 7 and 8 days after subconjunctival placement of a solid drug delivery system made of 10.2% rapamycin and 89.8% PVP K90.

FIG. 4 depicts the level of rapamycin in the vitreous (ng/ml), retina choroid and sclera of rabbit eyes at 14, 42, 63 and 91 days after subconjunctival placement of a solid drug delivery system made of 45.13% rapamycin, 40.03% PVP K90, 9.7% Eudragit RL100, and 5.14% PEG400.

FIG. 5 depicts the level of rapamycin in the aqueous humor (ng/ml) of rabbit eyes at 25, 35 and 37 days after subconjunctival placement of a solid drug delivery system with a backing, wherein the solid drug delivery system was made of 19.33% rapamycin, 21.78% PVP K90, 24.56% PEG 400, and 34.33% ethanol.

### DETAILED DESCRIPTION

Described herein are solid drug delivery systems and methods relating to delivery of therapeutic agents to a subject, including but not limited to a human subject or to the eye of a subject. These solid drug delivery systems and methods may be used for the treatment, prevention, inhibition, delaying onset of, or causing regression of diseases and conditions of the eye including but not limited to diseases or conditions of the posterior segment, including but not limited to choroidal neovascularization; macular degeneration; age-related macular degeneration (“AMD”), including wet AMD and dry AMD; retinal angiogenesis; chronic uveitis; and other retinoproliferative conditions. In some variations, the solid drug delivery systems or methods described herein are used for the treatment of the aforementioned diseases or conditions of the eye.

Herein are described (1) solid drug delivery systems including solid drug delivery systems with extended delivery of one or more therapeutic agents, (2) the therapeutic agents that may be delivered to a subject, including but not limited to a human subject or an eye of a subject using the solid drug delivery systems and methods described herein, (3) diseases and conditions that may be treated, prevented, inhibited, onset delayed, or regression caused by delivery of the therapeutic agents, (4) methods of treatment, (5) routes of administration for delivery of solid drug delivery systems and methods, (6) treatment of CNV and wet AMD by delivery of rapamycin to a subject or to the eye of a subject using the solid drug delivery systems described herein, and (7) administration of one or more antiproliferative agents proximal to a glaucoma drainage device.

Solid Drug Delivery Systems for Delivery of Therapeutic Agents

In this section are described solid drug delivery systems. In some variations the solid drug delivery systems comprise a therapeutic agent described in the Therapeutic Agents section, including but not limited to rapamycin. Delivery of therapeutic agents using the solid drug delivery systems described herein may be used to treat, prevent, inhibit, delay the onset of, or cause the regression of the diseases and conditions described herein. The solid drug delivery systems described herein may comprise one or more than one therapeutic agent. Other solid drug delivery systems in addition to those explicitly described herein may be used.

The solid drug delivery systems described herein comprise a therapeutic agent component and an excipient component. In some variations, the solid drug delivery systems described herein are capable of extended delivery of a therapeutic agent to an eye of a subject. The therapeutic agent component may comprise one or more therapeutic agents. The excipient component may comprise one or more excipients. The excipient component may comprise one or more solid or liquid solvents. In some variations the solid drug delivery system further comprises one or more solubilizing agents, surfactants, stabilizing agents, adjuvants, release modifying agents, antioxidants, etc.

The therapeutic agent may be, for instance, between 0.05 to 99% w/w; between 0.1 to 70%; between 1 to 50%; between 1.5 to 25%; between 5 to 20%; between 8 to 15%; between 5 to 10%; between 8 to 15%; between 1 to 5%; between 30 to 40%; between 40 to 50%; between 50 to 60%; between 60 to 70%; or between 70 to 80% w/w. By “w/w” is meant the weight of a given component as compared to the total weight of the final formulation.

The excipient component may be, for instance, between 5 to 99.9% of the total weight of the solid drug delivery system; between 10 to 90%; between 5 to 50%; between 1.5 to 25%; between 5 to 20%; between 8 to 15%; between 5 to 10%; between 8 to 15%; between 1 to 5%; between 30 to 40%; between 40 to 50%; between 50 to 60%; between 60 to 70%; between 70 to 80%; between 80 to 90%; or between 90 to 99.9%. The solid drug delivery systems may optionally further comprise surfactants, stabilizing agents, adjuvants, antioxidants, etc., between 0 and 40% by weight of the total.

The term “about,” as used herein, generally refers to the level of accuracy that is obtained when the methods described herein, such as the methods in the examples, are used. However, by “about” a certain amount of a component of a formulation is meant 90-110% of the amount stated.

The solid drug delivery systems described herein may be used to deliver amounts of the therapeutic agents effective for treating, preventing, inhibiting, delaying on set of, or causing the regression of the diseases and conditions described in the Diseases and Conditions section. In some variations the solid drug delivery systems described herein deliver one or more therapeutic agents over an extended period of time.

Generally, the therapeutic agent may be formulated in any solid drug delivery system capable of delivery of a therapeutically effective amount of the therapeutic agent to a subject or to the eye of a subject for the desired delivery period.

Excipients

The solid drug delivery systems described herein may comprise an excipient component. The excipient component may comprise one or more excipients. An “excipient,” as used herein, is any substance in the solid drug delivery system other than the therapeutic agent. Excipients may, for instance, aid in manufacturing the solid drug delivery system, assist in solubilizing the therapeutic agent, enhance the stability both prior and subsequent to placement of the solid drug delivery system, modify the delivery of the therapeutic agent to a target tissue, enhance transport through or to a tissue, or add color and flavor to the solid drug delivery system.

In some variations the excipient component may comprise one or more of solvents, surfactants, stabilizing agents, adjuvants, release modifying agents, antioxidants, etc. Note that there is overlap between categories of excipients, such as that are solvents, stabilizers, solubilizing agents or surfactants, and the same component can carry out more than one role. For example, polyvinylpyrrolidone (“PVP”) may be characterized by those of skill in the art as either a stabilizing agent or a solvent.

In some variations, the excipient component comprises a solvent component. The solvent may comprise one or more solvents. The solvent may be a solid or a liquid solvent. Any of the solvents described herein may be used in the excipient component.

In some variations, the solvent is polyethylene glycol. Polyethylene glycol is known by various names and is available in various preparations, including but not limited to macrogols, macrogel 400, macrogel 1500, macrogel 4000, macrogel 6000, macrogel 20000, macrogola, breox PEG; carbowax; carbowax sentry; Hodag PEG; Lipo; Lipoxol; Lutrol E; PEG; Pluriol E; polyoxyethylene glycol, and α-Hydro-ω-hydroxy-poly(oxy-1,2-ethanediyl). In some variations, the solvent component comprises a liquid polyethylene glycol. In some variations, the solvent component comprises a low molecular weight polyethylene glycol. In some variations, the solvent component comprises PEG 300 or PEG 400.

In some variations, the solvent is substantially absent from the solid drug delivery system after the solid drug delivery system is prepared. As one non-limiting example, a solvent may be added to the therapeutic agent, then during or after the processing removed. In some variations the solvent is substantially absent from the solid drug delivery system after its preparation, and the solid drug delivery system is a solid drug delivery system.

In some variations, the excipient component comprises a solubilizing agent component. The solubilizing agent component may comprise one or more solubilizing agents. Any of the solubilizing agents described herein may be used in the excipient component. In some variations the solubilizing agent is a surfactant.

In some variations, the excipient component comprises a stabilizing agent component. The stabilizing agent component may comprise one or more plasticizing agents. Any stabilizing agents may be used in the excipient component. In some variations, the stabilizing agent component comprises cross-linked or non-cross-linked polyvinylpyrrolidone (PVP).

In some variations, the excipient is a polyvinylpyrrolidone. Polyvinylpyrrolidone is known by various names and is available in various preparations, including but not limited to povidone, povidonum, kollidon; plasdone; poly[1-(2-oxo-1-pyrrolidinyl)ethylene]; polyvidone; PVP; 1-vinyl-2-pyrrolidinone polymer, and 1-Ethenyl-2-pyrrolidinone homopolymer. In some variations, the PVP is PVP K-90.

In some variations, the excipient component comprises a release modifying agent. In some variations, the release modifying agent is a film-forming polymer component. The film-forming polymer component may comprise one or more film-forming polymers. Any film-forming polymer may be used in the excipient component. In some variations, the film-forming polymer component comprises a water insoluble film forming polymer. In some variations, the film-forming polymer component comprises an acrylic polymer, including but not limited to polymethacrylate, including but not limited to Eudragit RL.

In some variations, the excipient is a polyacrylate. In some variations, the polyacrylate is a polymethacrylate. Polymethacrylates are known by various names and are available in various preparations, including but not limited to polymeric methacrylates, methacrylic acid-ethyl acrylate copolymer (1:1), methacrylic acid-ethyl acrylate copolymer (1:1) dispersion 30 percent, methacrylic acid-methyl methacrylate copolymer (1:1), methacrylic acid-methyl methacrylate copolymer (1:2), acidum methacrylicum et ethylis acrylas polymerisatum 1:1, acidum methacrylicum et ethylis acrylas polymerisatum 1:1 dispersio 30 per centum, acidum methacrylicum et methylis methacrylas polymerisatum 1:1, acidum methacrylicum et methylis methacrylas polymerisatum 1:2, USPNF: ammonio methacrylate copolymer, methacrylic acid copolymer, methacrylic acid copolymer dispersion.

In order to determine whether a potential agent may be used as an excipient in the solid drug delivery systems described herein, one of skill in the art may mix any of the therapeutic agents described herein, including but not limited to rapamycin, with any of the potential excipient components or agents as described herein, or any other excipient known in the art. The resulting solid drug delivery system may be placed in an appropriate animal model, including but not limited to placement in or proximal to the sclera or the area between the sclera and the conjunctiva of a rabbit eye, and average levels of therapeutic agent may monitored for an extended period of time.

Solvents for Therapeutic Agents

One solid drug delivery system that may be used is a solid drug delivery system comprising a solvent component.

In some variations any solvent may be used in which the therapeutic agent dissolves. In some variations the solvent is aqueous. In some variations the solvent is non-aqueous. An “aqueous solvent” is a solvent that contains at least about 50% water.

In some variations the solvent is a solid solvent and the resulting solution is a solid solution. In some variations, any solid solvent is used wherein the therapeutic agent, when combined with the solvent and placed in the subconjunctiva of a rabbit eye, gives extended release of the therapeutic agent as described herein. In some variations, the solvent and the therapeutic agent are mixed by blending, mixing, mechanical manipulation, precipitation, or some other method used in the art.

Generally, any concentration of therapeutic agent that has the desired effect can be used. The solvent component may be a single solvent or may be a mixture of solvents. Solvents and types of solutions are well known to those versed in such drug delivery technologies. See for example, Remington: The Science and Practice of Pharmacy, Twentieth Edition, Lippincott Williams & Wilkins; 20th edition (Dec. 15, 2000); Ansel's Pharmaceutical Dosage Forms and Drug Delivery Systems, Eighth Edition, Lippincott Williams & Wilkins (August 2004); Strickley, Solubilizing Excipeints in Oral and Injectable Formulations, Pharmaceutical Research, Vol. 21, No. 2, February 2004.

As noted previously, some solvents may also serve as solubilizing agents.

The solvent may remain in the solid drug delivery system or be removed after processing of the solid drug delivery system or placement of the solid drug delivery system in or proximal to the eye of the subject.

Solvents that may be used include but are not limited to DMSO, ethanol, methanol, isopropyl alcohol; castor oil, propylene glycol, polysorbate 80, benzyl alcohol, triacetin, diacetin, corn oil, ethyl lactate, glycerol formal, ethoxy diglycol (Transcutol, Gattefosse), tryethylene glycol dimethyl ether (Triglyme), dimethyl isosorbide (DMI), γ-butyrolactone, N-Methyl-2-pyrrolidinone (NMP), and polyglycolated capryl glyceride (Labrasol, Gattefosse) combinations of any one or more of the foregoing, or analogs or derivatives of any one or more of the foregoing.

In some variations, the solvent is glycerin, dimethylsulfoxide, N-methylpyrrolidone, dimethyl acetamide (DMA), dimethyl formamide, glycerol formal, ethoxy diglycol, triethylene glycol dimethyl ether, triacetin, diacetin, corn oil, acetyl triethyl citrate (ATC), ethyl lactate, polyglycolated capryl glyceride, γ butyrolactone, dimethyl isosorbide, benzyl alcohol, ethanol, isopropyl alcohol, polyethylene glycol of various molecular weights, including but not limited to PEG 300 and PEG 400, or propylene glycol, combinations of any one or more of the foregoing, or analogs or derivatives of any one or more of the foregoing.

In some variations, the solvent is a polyethylene glycol. Polyethylene glycol is known by various names and is available in various preparations, including but not limited to macrogels, macrogel 400, macrogel 1500, macrogel 4000, macrogel 6000, macrogel 20000, macrogola, breox PEG; carbowax; carbowax sentry; Hodag PEG; Lipo; Lipoxol; Lutrol E; PEG; Pluriol E; polyoxyethylene glycol, and α-Hydro-ω-hydroxy-poly(oxy-1,2-ethanediyl).

In some variations the polyethylene glycol is a liquid PEG, and is one or more of PEG 300 or PEG 400.

Other solvents include an amount of a C₆-C₂₄ fatty acid sufficient to solubilize a therapeutic agent.

Phospholipid solvents may also be used, such as lecithin, phosphatidylcholine, or a mixture of various diglycerides of stearic, palmitic, and oleic acids, linked to the choline ester of phosphoric acid; hydrogenated soy phosphatidylcholine (HSPC), distearoylphosphatidylglycerol (DSPG), L-α-dimyristoylphosphatidylcholine (DMPC), L-α-dimyristoylphosphatidylglycerol (DMPG).

Further examples of solvents include, for example, components such as alcohols, propylene glycol, polyethylene glycol of various molecular weights, propylene glycol esters, propylene glycol esterified with fatty acids such as oleic, stearic, palmic, capric, linoleic, etc; medium chain mono-, di-, or triglycerides, long chain fatty acids, naturally occurring oils, and a mixture thereof. The oily components for the solvent system include commercially available oils as well as naturally occurring oils. The oils may further be vegetable oils or mineral oils. The oils can be characterized as non-surface active oils, which typically have no hydrophile lipophile balance value. Commercially available substances comprising medium chain triglycerides include, but are not limited to, Captex 100, Captex 300, Captex 355, Miglyol 810, Miglyol 812, Miglyol 818, Miglyol 829, and Dynacerin 660. Propylene glycol ester compositions that are commercially available encompass Captex 200 and Miglyol 840, and the like. The commercial product, Capmul MCM, comprises one of many possible medium chain mixtures comprising monoglycerides and diglycerides.

Other solvents include naturally occurring oils such as peppermint oil, and seed oils. Exemplary natural oils include oleic acid, castor oil, safflower seed oil, soybean oil, olive oil, sunflower seed oil, sesame oil, and peanut oil. Soy fatty acids may also be used. Examples of fully saturated non-aqueous solvents include, but are not limited to, esters of medium to long chain fatty acids (such as fatty acid triglycerides with a chain length of about C₆ to about C₂₄). Hydrogenated soybean oil and other vegetable oils may also be used. Mixtures of fatty acids may be split from the natural oil (for example coconut oil, palm kernel oil, babassu oil, or the like) and refined. In some embodiments, medium chain (about C₈ to about C₁₂) triglycerides, such as caprilyic/capric triglycerides derived from coconut oil or palm seed oil, may be used. Medium chain mono- and diglycerides may also be used. Other fully saturated non-aqueous solvents include, but are not limited to, saturated coconut oil (which typically includes a mixture of lauric, myristic, palmitic, capric and caproic acids), including those sold under the Miglyol™ trademark from Huls and bearing trade designations 810, 812, 829 and 840). Also noted are the NeoBee™ products sold by Drew Chemicals. Non-aqueous solvents include isopropyl myristate. Examples of synthetic oils include triglycerides and propylene glycol diesters of saturated or unsaturated fatty acids having 6 to 24 carbon atoms such as, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, octadecanoic (stearic), nonadecanoic, heptadecanoic, eicosanoic, heneicosanoic, docosanoic and lignoceric acids, and the like. Examples of unsaturated carboxylic acids include oleic, linoleic and linolenic acids, and the like. The non-aqueous solvent can comprise the mono-, di- and triglyceryl esters of fatty acids or mixed glycerides and/or propylene glycol mono- or diesters wherein at least one molecule of glycerol has been esterified with fatty acids of varying carbon atom length. A non-limiting example of a “non-oil” useful as a solvent is polyethylene glycol.

Exemplary vegetable oils include cottonseed oil, corn oil, sesame oil, soybean oil, olive oil, fractionated coconut oil, peanut oil, sunflower oil, safflower oil, almond oil, avocado oil, palm oil, palm kernel oil, babassu oil, beechnut oil, linseed oil, rape oil and the like. Mono-, di-, and triglycerides of vegetable oils, including but not limited to corn, may also be used.

Polyvinyl pyrrolidone (PVP), cross-linked or not, may also be used as a solvent. Further solvents include but are not limited to C₆-C₂₄ fatty acids, oleic acid, Imwitor 742, Capmul, F68, F68 (Lutrol), PLURONICS including but not limited to PLURONICS F108, F127, and F68, Poloxamers, Jeffamines), Tetronics, F127; cyclodextrins such as α-cyclodextrin, β-cyclodextrin, hydroxypropyl-β-cyclodextrin, sulfobutylether-β-cyclodextrin (Captisol); carboxymethylcellulose (CMC), polysorbitan 20, Cavitron, polyethylene glycol of various molecular weights including but not limited to PEG 300 and PEG 400.

Beeswax and d-α-tocopherol (Vitamin E) may also be used as solvents.

Solvents for use in the solid drug delivery systems can be determined by a variety of methods known in the art, including but not limited to (1) theoretically estimating their solubility parameter values and choosing the ones that match with the therapeutic agent, using standard equations in the field; and (2) experimentally determining the saturation solubility of therapeutic agent in the solvents, and choosing the ones that exhibit the desired solubility.

Solubilization of Rapamycin

Where the therapeutic agent is rapamycin, solvents that may be used for making solid drug delivery systems comprising rapamycin include but are not limited to any solvent described herein, including but not limited to any one or more of DMSO, glycerin, ethanol, methanol, isopropyl alcohol; castor oil, propylene glycol, polyvinylpropylene, glycerin, polysorbate 80, benzyl alcohol, dimethyl acetamide (DMA), dimethyl formamide (DMF), glycerol formal, ethoxy diglycol (Transcutol, Gattefosse), tryethylene glycol dimethyl ether (Triglyme), dimethyl isosorbide (DMI), γ-butyrolactone, N-Methyl-2-pyrrolidinone (NMP), polyethylene glycol of various molecular weights, including but not limited to PEG 300 and PEG 400, and polyglycolated capryl glyceride (Labrasol, Gattefosse).

Further solvents include but are not limited to C₆-C₂₄ fatty acids, oleic acid, Imwitor 742, Capmul, F68, F68 (Lutrol), PLURONICS including but not limited to PLURONICS F108, F127, and F68, Poloxamers, Jeffamines), Tetronics, F127, beta-cyclodextrin, CMC, polysorbitan 20, Cavitron, softigen 767, captisol, and sesame oil.

Other methods that may be used to dissolve rapamycin are described in Solubilization of Rapamycin, P. Simamora et al. Int'l J. Pharma 213 (2001) 25-29, the contents of which is incorporated herein in its entirety.

Many other solvents are possible. Those of ordinary skill in the art will find it routine to identify which solvents may be used for rapamycin.

Release-Modifying Agents

In some variations, the release modifying agent accelerates the release rate of the therapeutic agent from the solid drug delivery system. In some variations, the release modifying agent slows the release rate of the therapeutic agent from the solid drug delivery system.

In some variations, the release modifying agent is a film-forming polymer component. The film-forming polymer component may comprise one or more film-forming polymers. Any film-forming polymer may be used in the excipient component. In some variations, the film-forming polymer component comprises a water insoluble film forming polymer. In some variations, the film-forming polymer component comprises an acrylic polymer.

In some variations, the release modifying agent is polymethacrylate. Polymethacrylates are known by various names and are available in various preparations, including but not limited to polymeric methacrylates, methacrylic acid-ethyl acrylate coploymer (1:1), methacrylic acid-ethyl acrylate coploymer (1:1) dispersion 30 percent, methacrylic acid-methyl methacrylate copolymer (1:1), methacrylic acid-methyl methacrylate copolymer (1:2), acidum methacrylicum et ethylis acrylas polymerisatum 1:1, acidum methacrylicum et ethylis acrylas polymerisatum 1:1 dispersio 30 per centum, acidum methacrylicum et methylis methacrylas polymerisatum 1:1, acidum methacrylicum et methylis methacrylas polymerisatum 1:2, USPNF: ammonio methacrylate copolymer, methacrylic acid copolymer, methacrylic acid copolymer dispersion. In some variations, the polymethacrylate is Eudragit RL.

Stabilizing Agents

The excipient component of the solid drug delivery systems described herein may comprise stabilizers. Stabilizers that may be used in the solid drug delivery systems described herein include but are not limited to agents that (1) improve the compatibility of excipients with the encapsulating materials such as gelatin, (2) improve the stability (e.g. prevent crystal growth) of a therapeutic agent including but not limited to rapamycin and/or rapamycin derivatives, and/or (3) improve solid drug delivery system stability.

Stabilizers include but are not limited to fatty acids, fatty alcohols, alcohols, long chain fatty acid esters, long chain ethers, hydrophilic derivatives of fatty acids, polyvinylpyrrolidones, polyvinylethers, polyvinyl alcohols, hydrocarbons, hydrophobic polymers, moisture-absorbing polymers, and combinations thereof. Amide analogues of the above stabilizers can also be used. The chosen stabilizer may change the hydrophobicity of the solid drug delivery system (e.g. oleic acid, waxes), or improve the mixing of various components in the solid drug delivery system (e.g. ethanol), control the moisture level in the formula (e.g. PVP), control the mobility of the phase (substances with melting points higher than room temperature such as long chain fatty acids, alcohols, esters, ethers, amides etc. or mixtures thereof; waxes), and/or improve the compatibility of the formula with encapsulating materials (e.g. oleic acid or wax). Some of these stabilizers may be used as solvents/co-solvents (e.g. ethanol). Stabilizers may be present in sufficient amount to inhibit the therapeutic agent's (such as rapamycin's) crystallization.

Examples of stabilizers include, but are not limited to, saturated, monoenoic, polyenoic, branched, ring-containing, acetylenic, dicarboxylic and functional-group-containing fatty acids such as oleic acid, caprylic acid, capric acid, caproic acid, lauric acid, myristic acid, palmitic acid, stearic acid, behenic acid, linoleic acid, linolenic acid; eicosapentaenoic Acid (EPA), docosahexaenoic acid, De-Hydroabietic Acid; fatty alcohols such as stearyl alcohol, cetyl alcohol, ceteryl alcohol; other alcohols such as ethanol, isopropyl alcohol, butanol; long chain fatty acid esters, ethers or amides such as glyceryl stearate, cetyl stearate, oleyl ethers, stearyl ethers, cetyl ethers, oleyl amides, stearyl amides; hydrophilic derivatives of fatty acids such as polyglyceryl fatty acids, polyethylene glycol fatty acid esters; polyvinylpyrrolidones, polyvinylalcohols, waxes etc.

In some variations, the stabilizing agent is polyvinylpyrrolidone. Polyvinylpyrrolidone is known by various names and is available in various preparations, including but not limited to povidone, povidonum, kollidon; plasdone; poly[1-(2-oxo-1-pyrrolidinyl)ethylene]; polyvidone; PVP; 1-vinyl-2-pyrrolidinone polymer, and 1-Ethenyl-2-pyrrolidinone homopolymer.

Gelling Agents

The excipient component of the solid drug delivery systems described herein may comprise a gelling agent that alters the texture of the final solid drug delivery system through formation of a gel.

Gelling agents that may be used include but are not limited to carrageenan, cellulose gel, colloidal silicon dioxide, gelatin, propylene carbonate, carbonic acid, alginic acid, agar, carboxyvinyl polymers or carbomers and polyacrylamides, acacia, ester gum, guar gum, gum arabic, ghatti, gum karaya, tragacanth, terra, pectin, tamarind seed, larch arabinogalactan, alginates, locust bean, xanthan gum, starch, veegum, tragacanth, polyvinyl alcohol, gellan gum, hydrocolloid blends, and povidone.

Adjuvants

The excipient component of the solid drug delivery systems described herein may comprise one or more adjuvants appropriate for the indicated route of administration or placement. Adjuvants with which the therapeutic agent may be admixed with include but are not limited to lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. When a solubilized solid drug delivery system is required the therapeutic agent may be in a solvent or solubilizing agent including but not limited to polyethylene glycol, propylene glycol, carboxymethyl cellulose colloidal solutions, methanol, ethanol, DMSO, corn oil, peanut oil, cottonseed oil, sesame oil, tragacanth gum, and/or various buffers. Other adjuvants and modes of administration are well known in the pharmaceutical art and may be used in the practice of the methods and solid drug delivery systems described herein. The carrier or diluent may include time delay material, such as glyceryl monostearate or glyceryl distearate alone or with a wax, or other materials well known in the art. The solid drug delivery system for use as described herein may also comprise gel formulations, erodible and non-erodible polymers, micropsheres, and liposomes.

Other adjuvants and excipients that may be used include but are not limited to C8-C10 fatty acid esters such as softigen 767, polysorbate 80, Pluronics, Tetronics, Miglyol, and Transcutol.

Additives and Diluents

The excipient component of the solid drug delivery systems described herein may comprise additives or diluents, such as those normally utilized in the pharmaceutical arts. These include thickening, granulating, dispersing, flavoring, sweetening, coloring, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like. Exemplary preservatives include, but are not limited to, benzylalcohol, ethylalcohol, benzalkonium chloride, phenol, chlorobutanol, and the like. Some useful antioxidants provide oxygen or peroxide inhibiting agents for the solid drug delivery system and include, but are not limited to, butylated hydroxytoluene, butylhydroxyanisole, propyl gallate, ascorbic acid palmitate, α-tocopherol, and the like. Thickening agents, such as lecithin, hydroxypropylcellulose, aluminum stearate, and the like, may improve the texture of the solid drug delivery system.

In addition, a viscous polymer may be added to the suspension, assisting the localization in the eye, including but not limited to the sclera, and ease of placement and handling. In some uses of the solid drug delivery system, a pocket in the sclera may be surgically formed to receive an injection or placement of the solid drug delivery systems. Particles of therapeutic agent substance for forming a suspension can be produced by known methods including but not limited to via ball milling, for example by using ceramic beads. For example, a Cole Parmer ball mill such as Labmill 8000 may be used with 0.8 mm YTZ ceramic beads available from Tosoh or Norstone Inc.

Many other solvents are possible. Those of ordinary skill in the art will find it routine to identify solvents for rapamycin given the teachings herein.

Solubilizing Agents

The excipient component of the solid drug delivery systems described herein may comprise one or more solubilizing agents. Generally, any solubilizing agent or combination of solubilizing agents may be used in the solid drug delivery systems described herein.

In some variations, the solubilizing agent is a surfactant or combination of surfactants. Many solubilizing agents and surfactants are possible. In some variations, combinations of solubilizing agents or surfactants, including but not limited to combinations of various types of solubilizing agents or surfactants, may also be used. For instance, surfactants which are nonionic, anionic (i.e. soaps, sulfonates), cationic (i.e. CTAB), zwitterionic, polymeric or amphoteric may be used.

In some variations, a solubilizing agent or surfactant for use in the solid drug delivery systems described herein is determined by mixing a putative solubilizing agent or surfactant with a solid drug delivery system as described herein, and observing the characteristics of the solid drug delivery system after placement in a subject.

Examples of surfactants include but are not limited to fatty acid esters or amides or ether analogues, or hydrophilic derivatives thereof; monoesters or diesters, or hydrophilic derivatives thereof; or mixtures thereof; monoglycerides or diglycerides, or hydrophilic derivatives thereof; or mixtures thereof; mixtures having enriched mono- or/and diglycerides, or hydrophilic derivatives thereof; surfactants with a partially derivatized with a hydrophilic moiety; monoesters or diesters or multiple-esters of other alcohols, polyols, saccharides or oligosaccharides or polysaccharides, oxyalkylene oligomers or polymers or block polymers, or hydrophilic derivatives thereof, or the amide analogues thereof; fatty acid derivatives of amines, polyamines, polyimines, aminoalcohols, aminosugars, hydroxyalkylamines, hydroxypolyimines, peptides, polypeptides, or the ether analogues thereof.

Hydrophilic Lipophilic Balance (“HLB”) is an expression of the relative simultaneous attraction of a surfactant for water and oil (or for the two phases of the emulsion system being considered).

Surfactants are characterized according to the balance between the hydrophilic and lipophilic portions of their molecules. The hydrophilic-lipophilic balance (HLB) number indicates the polarity of the molecule in an arbitrary range of 1-40, with the most commonly used emulsifiers having a value between 1 and 20. The HLB increases with increasing hydrophilicity.

Surfactants that may be used include but are not limited to those with an HLB greater than 10, 11, 12, 13 or 14. Examples of surfactants include polyoxyethylene products of hydrogenated vegetable oils, polyethoxylated castor oils or polyethoxylated hydrogenated castor oil, polyoxyethylene-sorbitan-fatty acid esters, polyoxyethylene castor oil derivatives and the like, for example, Nikkol HCO-50, Nikkol HCO-35, Nikkol HCO-40, Nikkol HCO-60 (from Nikko Chemicals Co. Ltd.); Cremophor (from BASF) such as Cremophor RH40, Cremophor RH60, Cremophor EL, TWEENs (from ICI Chemicals) e.g., TWEEN 20, TWEEN 21, TWEEN 40, TWEEN 60, TWEEN 80, TWEEN 81, Cremophor RH 410, Cremophor RH 455 and the like.

The surfactant component may be selected from compounds having at least one ether formed from at least 1 to 100 ethylene oxide units and at least one fatty alcohol chain having from at least 12 to 22 carbon atoms; compounds having at least one ester formed from at least about 1 to 100 ethylene oxide units and at least one fatty acid chain having from at least 12 to 22 carbon atoms; compounds having at least one ether, ester or amide formed from at least 1 to 100 ethylene oxide units and at least one vitamin or vitamin derivative; and combinations thereof consisting of no more than two surfactants.

Other examples of surfactants-include Lumulse GRH-40, TGPS, Polysorbate-80 (TWEEN-80), Polysorbate-20 (TWEEN-20), polyoxyethylene (20) sorbitan mono-oleate), glyceryl glycol esters, polyethylene glycol esters, polyglycolyzed glycerides, and the like, or mixtures thereof; polyethylene sorbitan fatty acid esters, polyoxyethylene glycerol esters, such as Tagat TO, Tagat L, Tagat I, tagat 12 and Tagat 0 (commercially available from Goldschmidt Chemical Co., Essen, Germany); ethylene glycol esters, such as glycol stearate and distearate; propylene glycol esters, such as propylene glycol myristate; glyceryl esters of fatty acids, such as glyceryl stearates and monostearates; sorbitan esters, such as spans and TWEENs; polyglyceryl esters, such as polyglyceryl 4-oleate; fatty alcohol ethoxylates, such as Brij type emulsifiers; ethoxylated propoxylated block copolymers, such as poloxamers; polyethylene glycol esters of fatty acids, such as PEG 300 linoleic glycerides or Labrafil 2125 CS, PEG 300 oleic glycerides or Labrafil M 1944 CS, PEG 400 caprylic/capric glycerides or Labrasol, and PEG 300 caprylic/capric glycerides or Softigen 767; cremophors, such as Cremophor E, polyoxyl 35 castor oil or Cremophor EL, Cremophor EL-P, Cremophor RH 4OP, polyoxyl 40 hydrogenated castor oil, Cremophor RH40; polyoxyl 60 hydrogenated castor oil or Cremophor RH 60, glycerol monocaprylate/caprate, such as Campmul CM 10; polyoxyethylated fatty acids (PEG-stearates, PED-laurates, Brij®), polyoxylated glycerides of fatty acid, polyoxylated glycerol fatty acid esters i.e. Solutol HS-15; PEG-ethers (Mirj®), sorbitan derivatives (TWEENs), sorbitan monooleate or Span 20, aromatic compounds (Tritons®), PEG-glycerides (PECEOL™), PEG-PPG (polyethylene glycol-polypropylene glycol) copolymers (PLURONICS including but not limited to PLURONICS F108, F127, and F68, Poloxamers, Jeffamines), Tetronics, Polyglycerines, PEG-tocopherols, PEG-LICOL 6-oleate; propylene glycol derivatives, sugar and polysaccharide alkyl and acyl derivatives (octylsucrose, sucrose stearate, laurolydextran etc.) and/or a mixture thereof; surfactants based on an oleate or laureate ester of a polyalcohol copolymerized with ethylene oxide; Labrasol Gelucire 44/14; polyoxytheylene stearates; saturated polyglycolyzed glycerides; or poloxamers; all of which are commercially available. Polyoxyethylene sorbitan fatty acid esters can include polysorbates, for example, polysorbate 20, polysorbate 40, polysorbate 60, and polysorbate 80. Polyoxyethylene stearates can include polyoxyl 6 stearate, polyoxyl 8 stearate, polyoxyl 12 stearate and polyoxyl 20 stearate. Saturated polyglycolyzed glycerides are, for example, GELUCIRE 44/14 or GELUCIRE™ 50/13 (Gattefosse, Westwood, N.J., U.S.A.). Poloxamers used herein include poloxamer 124 and poloxamer 188.

Surfactants include d-α-tocopheryl polyethylene glycol 1000 succinate (TPGS), polyoxyl 8 stearate (PEG 400 monostearate), polyoxyl 40 stearate (PEG 1750 monostearate) and peppermint oil.

In some variations, surfactants having an HLB lower than 10 are used. Such surfactants may optionally be used in combination with other surfactants as co-surfactants. Examples of some surfactants, mixtures, and other equivalent compositions having an HLB less than or equal to 10 are propylene glycols, glyceryl fatty acids, glyceryl fatty acid esters, polyethylene glycol esters, glyceryl glycol esters, polyglycolyzed glycerides and polyoxyethyl steryl ethers. Propylene glycol esters or partial esters form the composition of commercial products, such as Lauroglycol FCC, which contains propylene glycol laureate. The commercially available excipient Maisine 35-1 comprises long chain fatty acids, for example glyceryl linoleate. Products, such as Acconon E, which comprise polyoxyethylene stearyl ethers, may also be used. Labrafil M 1944 CS is one example of a surfactant wherein the composition contains a mixture of glyceryl glycol esters and polyethylene glycol esters.

Solubilizing Agents for Rapamycin

Many solubilizing agents or surfactants may be used for rapamycin, including but not limited to any solubilizing agent described herein, including but not limited to the solubilizing agents in this section.

In some variations the solubilizing agent is a surfactant. Nonlimiting examples of surfactants that may be used for rapamycin include but are not limited to surfactants with an HLB greater than 10, 11, 12, 13 or 14. One nonlimiting example is Cremophor EL. In some variations, the surfactant may be a polymeric surfactant including but not limited to PLURONICS F108, F127, and F68, and Tetronics. As noted above, some solubilizing agents may also serve as solvents. Those of ordinary skill in the art will find it routine to identify which surfactants may be used for rapamycin given the teachings herein.

Viscosity Modifying Agents

The solid drug delivery systems described herein may be placed in combination with or further comprise a viscosity modifying agent.

One exemplary viscosity modifying agent that may be used is hyaluronic acid. Hyaluronic acid is a glycosaminoglycan. It is made of a repetitive sequence of glucuronic acid and glucosamine. Hyaluronic acid is present in many tissues and organs of the body, and contributes to the viscosity and consistency of such tissues and organs. Hyaluronic acid is present in the eye, including the vitreous of the eye, and along with collagen contributes to the viscosity thereof. The solid drug delivery systems described herein may further comprise or be administered with hyaluronic acid.

Other nonlimiting examples of viscosity modifying agents include polyalkylene oxides, glycerol, carboxymethyl cellulose, sodium alginate, chitosan, dextran, dextran sulfate and collagen. These viscosity modifying agents can be chemically modified.

Other viscosity modifying agents that may be used include but are not limited to carrageenan, cellulose gel, colloidal silicon dioxide, gelatin, propylene carbonate, carbonic acid, alginic acid, agar, carboxyvinyl polymers or carbomers and polyacrylamides, acacia, ester gum, guar gum, gum arabic, ghatti, gum karaya, tragacanth, terra, pectin, tamarind seed, larch arabinogalactan, alginates, locust bean, xanthan gum, starch, veegum, tragacanth, polyvinyl alcohol, gellan gum, hydrocolloid blends, and povidone. Other viscosity modifying agents known in the art can also be used, including but not limited to sodium carboxymethyl cellulose, algin, carageenans, galactomannans, hydropropyl methyl cellulose, hydroxypropyl cellulose, polyethylene glycol, polyvinylpyrrolidone, sodium carboxymethyl chitin, sodium carboxymethyl dextran, sodium carboxymethyl starch, xanthan gum, and zein.

Other Components of Formulations

The formulations described herein may further comprise various other components such as stabilizers, for example. Stabilizers that may be used in the formulations described herein include but are not limited to agents that will (1) improve the compatibility of excipients with the encapsulating materials such as gelatin, (2) improve the stability (e.g. prevent crystal growth of a therapeutic agent such as rapamycin) of a therapeutic agent such as rapamycin and/or rapamycin derivatives, and/or (3) improve formulation stability. Note that there is overlap between components that are stabilizers and those that are solvents, solubilizing agents or surfactants, and the same component can carry out more than one role.

Stabilizers may be selected from fatty acids, fatty alcohols, alcohols, long chain fatty acid esters, long chain ethers, hydrophilic derivatives of fatty acids, polyvinylpyrrolidones, polyvinylethers, polyvinyl alcohols, hydrocarbons, hydrophobic polymers, moisture-absorbing polymers, and combinations thereof. Amide analogues of the above stabilizers can also be used. The chosen stabilizer may change the hydrophobicity of the formulation (e.g. oleic acid, waxes), or improve the mixing of various components in the formulation (e.g. ethanol), control the moisture level in the formula (e.g. PVP), control the mobility of the phase (substances with melting points higher than room temperature such as long chain fatty acids, alcohols, esters, ethers, amides etc. or mixtures thereof; waxes), and/or improve the compatibility of the formula with encapsulating materials (e.g. oleic acid or wax). Some of these stabilizers may be used as solvents/co-solvents (e.g. ethanol). Stabilizers may be present in sufficient amount to inhibit the therapeutic agent's (such as rapamycin's) crystallization.

Examples of stabilizers include, but are not limited to, saturated, monoenoic, polyenoic, branched, ring-containing, acetylenic, dicarboxylic and functional-group-containing fatty acids such as oleic acid, caprylic acid, capric acid, caproic acid, lauric acid, myristic acid, palmitic acid, stearic acid, behenic acid, linoleic acid, linolenic acid, eicosapentaenoic acid (EPA), DHA; fatty alcohols such as stearyl alcohol, cetyl alcohol, ceteryl alcohol; other alcohols such as ethanol, isopropyl alcohol, butanol; long chain fatty acid esters, ethers or amides such as glyceryl stearate, cetyl stearate, oleyl ethers, stearyl ethers, cetyl ethers, oleyl amides, stearyl amides; hydrophilic derivatives of fatty acids such as polyglyceryl fatty acids, polyethylene glycol fatty acid esters; polyvinylpyrrolidones, polyvinylalcohols (PVAs), waxes, docosahexaenoic acid and de-hydroabietic acid etc.

The therapeutic agents for use as described herein, such as rapamycin, may be subjected to conventional pharmaceutical operations, such as sterilization and compositions containing the therapeutic agent may also contain conventional adjuvants, such as preservatives, stabilizers, wetting agents, emulsifiers, buffers etc. The therapeutic agents may also be formulated with pharmaceutically acceptable excipients for clinical use to produce a solid drug delivery system.

The therapeutic agents may be used to prepare a medicament to treat, prevent, inhibit, delay onset, or cause regression of any of the conditions described herein. In some variations, one or more therapeutic agents are used to prepare a medicament to treat any of the conditions described herein. In some variations, one or more therapeutic agents are used to prepare a medicament to prevent any of the conditions described herein.

A solid drug delivery system containing a therapeutic agent such as rapamycin may contain one or more adjuvants appropriate for the indicated route of administration. Adjuvants with which the therapeutic agent may be admixed with include but are not limited to lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol. When a solubilized formulation is required the therapeutic agent may be in a solvent including but not limited to polyethylene glycol of various molecular weights, propylene glycol, carboxymethyl cellulose colloidal solutions, methanol, ethanol, DMSO, corn oil, peanut oil, cottonseed oil, sesame oil, tragacanth gum, and/or various buffers. Other adjuvants and modes of administration are well known in the pharmaceutical art and may be used in the practice of the methods and solid drug delivery systems described herein. The carrier or diluent may include time delay material, such as glyceryl monostearate or glyceryl distearate alone or with a wax, or other materials well known in the art. The formulations for use as described herein may also include gel formulations, erodible and non-erodible polymers, microspheres, and liposomes. Other adjuvants and excipients that may be used include but are not limited to C₈-C₁₀ fatty acid esters such as softigen 767, polysorbate 80, PLURONICS, Tetronics, Miglyol, and Transcutol.

Additives and diluents normally utilized in the pharmaceutical arts can optionally be added to the solid drug delivery systems described herein. These include thickening, granulating, dispersing, flavoring, sweetening, coloring, and stabilizing agents, including pH stabilizers, other excipients, anti-oxidants (e.g., tocopherol, BHA, BHT, TBHQ, tocopherol acetate, ascorbyl palmitate, ascorbic acid propyl gallate, and the like), preservatives (e.g., parabens), and the like. Exemplary preservatives include, but are not limited to, benzylalcohol, ethylalcohol, benzalkonium chloride, phenol, chlorobutanol, and the like. Some useful antioxidants provide oxygen or peroxide inhibiting agents for the formulation and include, but are not limited to, butylated hydroxytoluene, butylhydroxyanisole, propyl gallate, ascorbic acid palmitate, α-tocopherol, and the like. Thickening agents, such as lecithin, hydroxypropylcellulose, aluminum stearate, and the like, may improve the texture of the formulation.

In some variations, the therapeutic agent is rapamycin, and the rapamycin is formulated as rapamune in solid form. In some variations, the rapamune is formulated as an oral dosage.

In addition, a viscous polymer may be added to the suspension, assisting the localization and ease of placement and handling. In some uses of the solid drug delivery systems, a pocket in the sclera may be surgically formed for placement of the solid drug delivery system. The hydrogel structure of the sclera can act as a rate-controlling membrane.

The solid drug delivery systems may conveniently be presented in unit dosage form and may be prepared by conventional pharmaceutical techniques. Such techniques include the step of bringing into association the therapeutic agent and the pharmaceutical carrier(s) or excipient(s). The formulations may be prepared by uniformly and intimately bringing into associate the active ingredient with liquid carriers or finely divided solid carriers or both, and then, if necessary, shaping the product.

In some variations, the formulations described herein are provided in one or more unit dose forms, wherein the unit dose form contains an amount of a solid drug delivery system described herein that is effective to treat or prevent the disease or condition for which it is being administered. In some variations, the solid drug delivery systems described herein are provided in one or more unit dose forms, wherein the unit dose form contains an amount of a rapamycin formulation described herein that is effective to treat or prevent the disease or condition for which it is being administered for a period of time.

In a further aspect, provided herein are kits comprising one or more unit dose forms as described herein. In some embodiments, the kit comprises one or more of packaging and instructions for use to treat one or more diseases or conditions, including but not limited to the diseases or conditions described herein. In some embodiments, the kit comprises any of one or more unit dose forms described herein in one or more sealed vessels or sealed packaging. In some embodiments, the kit comprises any of one or more sterile unit dose forms.

In some variations, the unit dose form is in a container, including but not limited to a sterile sealed container or packaging. In some variations the container is a vial, ampule, or low volume applicator.

Described herein are kits comprising one or more unit dose forms comprising one or more solid drug delivery systems. In some variations the kit comprises one or more containers with instructions for its use. In some variations a kit comprises one or more solid drug delivery systems in a container or packaging, wherein the solid drug delivery system comprises rapamycin, and the kit further comprises instructions for use of the solid drug delivery system in treating a disease or condition of the eye. In some variations, the solid drug delivery system is in a container and the container is in a secondary packaging.

Backed Solid Drug Delivery Systems

In some variations the solid delivery systems described herein comprise a backing. In some variations the backing is bioerodible. In some variations the backing is nonbioerodible. In some variations the backing is a combination of one or more bioerodible and one or more nonbioerodible materials.

In some variations, the solid drug delivery systems with a backing are designed to promote diffusion in a direction of choice.

In some variations a backed solid delivery system described herein includes an erodible implant, such as a disk, cylinder, fiber, or film comprising the active therapeutic agent, and a backing made of an erodible polymer that contains little or no therapeutic agent. The choice of the second erodible polymer can be such that elution of therapeutic agent from the implant in the direction of the second polymer is blocked or slowed, allowing for the therapeutic agent to be delivered primarily in one direction.

In some variations, the backing layer is at least partially impermeable to the therapeutic agent. By “at least partially permeable” is meant that the rate of the therapeutic agent exiting the drug delivery system through the backing is lower than the rate of the therapeutic agent exiting the drug delivery system through the portion without the backing.

In some variations, the backing layer is substantially impermeable to the therapeutic agent. In some variations, a non-erodible polymer is used as the blocking layer, and the backing layer is removed some period of time after placement in the subject.

As used herein, “substantially impermeable” means that a clinically insignificant amount of therapeutic agent passes through the substantially impermeable barrier. In some variations, the substantially impermeable barrier is for all practical purposes impermeable to the therapeutic agent.

In some variations of a solid drug delivery system with a backing, a suture is sandwiched between the solid drug delivery system and the backing to allow the structure to remain securely affixed to the sclera via the suture. In some variations the backing allows an amount of the therapeutic agent through that does not cause local toxic effects in the subject to which the solid drug delivery system is administered.

Generally, the backing can be made of any material that diminishes diffusion of the therapeutic agent into the tissues proximal to the backing as compared to diffusion into such tissues in the absence of the backing. In some variations the backing is not completely impermeable to the therapeutic agent but has such disparity of diffusion thereto that for practical purposes the vast majority of the drug elutes toward the scleral surface. The backing material may be impermeable or substantially impermeable to the therapeutic agent or may be semi-permeable or permeable to the therapeutic agent. In one backed polymer implant, the material of the therapeutic agent-containing solid drug delivery system and the backing are the same, and the concentration of the therapeutic agent in the therapeutic agent containing polymer is greater than the concentration in the backing. In one such implant, the backing initially contains substantially no therapeutic agent.

In some variations the backing is shaped and sized to hold a solid drug delivery system and reside in an ocular site. In some variations, the backing is in the shape of a thin, shallow saucer or cup. In some variations, the backing is made of a thermoplastic. In some variations, the backing is made of a polyetheretherketone (PEEK), including but not limited to Victrex K90.

In some variations, a formulation is prepared and placed in a backing before it has become solid; by way of nonlimiting example, the formulation is placed in a backing before one or more solvents has been evaporated off. Such variations may include but are not limited to those formulations shown in Table 2. Some formulations, prior to drying, are generally but need not be suspensions.

In some variations, the formulation is allowed to dry prior to placement in the subject. In some variations, the formulation is not dry upon placement in the subject.

Delivery by Solid Drug Delivery System with Delayed Release

One solid drug delivery system that may be used to deliver the therapeutic agent is a delayed release solid drug delivery system.

In one solid drug delivery system, the onset of therapeutic agent release is delayed for a period of time after the solid drug delivery system insertion into the eye. This delay allows for example, for time for the wound caused by the insertion of the solid drug delivery system to heal prior to therapeutic agent delivery. Such a delay is advantageous when the therapeutic agent itself inhibits wound healing. For example, therapeutic agents that inhibit fibroblastic proliferation, such as rapamycin, will inhibit wound healing. In one such delayed release solid drug delivery system that may be used, therapeutic agent release is delayed by coating the solid drug delivery system containing the therapeutic agent with a polymer that contains no or a lesser amount of a therapeutic agent but that will erode during a predetermined time. Thus, therapeutic agent release is delayed until a substantial portion of the polymer coating has eroded away. As used herein, a “substantial portion” of a substance refers to in excess of 80% of the substance. The polymer coating may be substantially impermeable to the therapeutic agent.

Given the teachings herein, one versed in delayed release technology will be able to identify other solid drug delivery systems that may be used to achieve the delayed release described herein.

Depending on the therapeutic agent being delivered and/or the diseases and conditions being treated or prevented this period of delay before delivery of the therapeutic agent commences may be 1 hour, 6 hours, 12 hours, 18 hours, 1 day, 2 days, 3 days, 4 days, 5 days, 6 days, 7 days, 8 days, 9 days, 10 days, 11 days, 12 days, 13 days, 14 days, 21 days, 28 days, 35 days, or 42 days. Other delay periods may be possible. Delayed release systems that may be used are known to people versed in the technology, and includes but is not limited to the use of a coating or reservoir.

Delivery by a Bioadhesive Solid Drug Delivery System

One delivery system that may be used is a solid drug delivery system in which the therapeutic agent is delivered by placement of a solid drug delivery system that includes a bioadhesive surface.

The bioadhesive surface of the solid drug delivery system allows the solid drug delivery system to be secured in place by adhesion to a biomaterial in the ocular region, including but not limited to adhesion to the outer scleral surface. The bioadhesive solid drug delivery system may be made of a bioadhesive polymer material or may be made of a non-bioadhesive polymer material that is coated with a bioadhesive material to form the bioadhesive surface. The preparation of solid drug delivery systems with bioadhesive surfaces is well known to those versed in the technology. See, for example, Bioadhesive any phase-change polymers for ocular drug delivery, J. Robinson et al., Advanced Drug Delivery Review, 16 (1995) 45-50, the contents of which is incorporated herein in its entirety.

Bioadhesive polymers that may be used include but are not limited to the following or any mixtures of the following: Polyvinyl pyrrolidone of various molecular weight, polyacrylic acid and copolymers of acrylic acid and acrylate esters, cross-linked polyacrylic acids (carbopols), celluloses (ethyl cellulose, methyl cellulose, microcrystalline cellulose, etc.,), cellulose derivatives (hydroxy ethyl cellulose, hydroxy propyl cellulose, hydroxypropyl methyl cellulose, carboxy methyl cellulose, etc.,), cellulose esters (cellulose acetate, cellulose phthalate, cellulose acetate phthalate, cellulose acetate butyrate, cellulose acetate propionate, etc.,), gums (gum arabica, tragacanth, gum acacia, gallen gum, xanthan gum, etc.,), hyaluronic acid and its derivatives, polyethylene oxides (polyox and derivatives, polyethylene glycol, and graft polymers of polyethylene oxides), chitosan and alginic acid.

The bioadhesive polymers may be mixed with suitable plasticizers to obtain a flexible film. Plasticizers that may be used include but are not limited to Propylene glycol, polypropylene glycol, polyethylene glycol, glycerol, glycerol esters (eg. glycerol monololeate), and esters of propylene glycol (eg. propylene glycol monolaurate), and water.

The bioadhesive polymers may be mixed with suitable wetting agents at a very low concentrations to improve surface contact when a bioadhesive solid drug delivery system is placed on the tissue: Wetting agents that may be used include but are not limited Surfactants: Cholesterol, tweens and spans, polysorbate 80, and pluronics.

The bioadhesive polymers may be mixed with suitable excipients, including but not limited to quickly dissolving water absorbent sugars/starches, such as mannitol, dextrose, lactose, maltodextrins. It is believed that because the tissues to which the solid drug delivery system will adhere possesses a certain amount of moisture, these sugars/starches will help absorb the moisture more quickly so that initial bioadhesion and contact is achieved more readily.

Shape Memory Solid Drug Delivery Systems

The solid drug delivery systems described herein may comprise a solid drug delivery system with shape-memory properties (a “shape memory solid drug delivery system”). A shape memory solid drug delivery system, as used herein, indicates a solid drug delivery system comprised of, e.g. a shape memory polymer, whose macroscopic shape may be processed or formed into a first shape, subsequently processed or formed into a second shape, and which upon exposure to a predetermined condition changes or reverts to a shape that is similar or identical to the first shape. The shape memory solid drug delivery system may be made of various polymers. For further information on shape-memory polymers, see Alteheld et al., Biodegradable, Amorphous Copolyester-Urethane Networks Having Shape-Memory Properties, Andew. Chem. Int. Ed. 44: 1188-1192 (2005), which is incorporated herein by reference in its entirety.

In some variations, a shape memory solid drug delivery system changes or reverts to a shape that is similar or identical to the first shape within 24, 20, 15, 10, 6, 4, 2, or 1 hours. In some variations, the shape memory solid drug delivery system changes or reverts to a shape that is similar or identical to the first shape within 45, 30, 20, or 10 minutes.

In some variations, a shape memory solid drug delivery system comprises a shape memory polymer wherein the second shape of the shape memory solid drug delivery system is smaller, more compact, or compressed relative to the first shape. In such a variation, the shape memory solid drug delivery system may be made smaller, more compact or compressed relative to the first shape in order to place the solid drug delivery system, including but not limited to via injection.

In some variations, the second shape of the shape memory solid drug delivery system has an overall more linear shape relative to the first shape. In such a variation, the shape memory solid drug delivery system may be made overall more linear relative to the first shape in order to place the solid drug delivery system, including but not limited to via injection.

In some variations, the shape memory solid drug delivery system, after placement in or proximal to the eye of a subject, changes or reverts to a shape that is similar or identical to the first shape.

In some variations, the shape memory solid drug delivery system is transparent or essentially transparent. In some variations the shape memory solid drug delivery system is bioerodible. In some variations the shape memory solid drug delivery system is nonbioerodible. In some variations, the shape memory solid drug delivery system is amorphous. In some variations the shape memory solid drug delivery system is prepared from star-shaped hydroxyl-telechelic co-oligoesters.

Extended Delivery of Therapeutic Agents Including Rapamycin

For treatment, prevention, inhibition, delaying the onset of, or causing the regression of certain diseases or conditions, it may be desirable to maintain delivery of a therapeutically effective amount of the therapeutic agent for an extended period of time. Depending on the disease or condition being treated, prevented, inhibited, having onset delayed, or being caused to regress this extended period of time may be at least 30 days, at least 60 days, at least 90 days, at least 120 days, at least 150 days, at least 180 days, at least 210 days, at least 240 days, at least 270 days, at least 300 days, at least 330 days, or at least 360 days. Generally, however, any extended period of delivery may be possible. A therapeutically effective amount of agent may be delivered for an extended period by a solid drug delivery system that maintains for the extended period a concentration of agent in a subject or an eye of a subject sufficient to deliver a therapeutically effective amount of agent for the extended time.

Delivery of a therapeutically effective amount of the therapeutic agent for an extended period may be achieved using application of one solid drug delivery system or may be achieved by application of two or more solid drug delivery systems, either at the same time or some period of time from one another. As a non-limiting example of such multiple applications, maintenance of the therapeutic amount of rapamycin for 3 months for treatment of wet AMD may be achieved by application of one solid drug delivery system delivering a therapeutic amount for 3 months or by sequential application of a plurality of solid drug delivery systems. The optimal dosage regime will depend on the therapeutic amount of the therapeutic agent needing to be delivered, the period over which it need be delivered, and the size of the system needed to satisfy these requirements. Those versed in such extended therapeutic agent delivery dosing will understand how to identify dosing regimes that may be used given the teachings provided herein.

Described herein are solid drug delivery systems showing in vivo delivery or clearance profiles with one or more of the following characteristics. The delivery or clearance profiles are for clearance of the therapeutic agent in vivo after placement of the solid drug delivery system in the area between the sclera and conjunctiva of a rabbit eye. The therapeutic agent may be any of the therapeutic agents as herein, including but not limited to rapamycin. The solid drug delivery system may be any solid drug delivery system described herein, including but not limited to the solid drug delivery system prepared in Example 1. The volume of a vitreous of a rabbit eye is approximately 30-40% of the volume of a vitreous of a human eye. The amount of therapeutic agent is measured using techniques as described in Example 2, but without limitation to the solid drug delivery system and therapeutic agent described in Example 2.

In some variations, the solid drug delivery systems described herein may have in vivo delivery to the vitreous profiles with the following described characteristics, where the delivery profiles are for delivery of therapeutic agent in vivo after placement of the solid drug delivery system into the area between the sclera and the conjunctiva of a rabbit eye.

“Average percentage in vivo” level or concentration means that an average concentration of therapeutic agent is obtained across multiple rabbit eyes for a given timepoint, and the average concentration of therapeutic agent at one timepoint is divided by the average concentration of therapeutic agent at another timepoint. In some variations of the average percentage in vivo levels, the therapeutic agent is rapamycin.

In some variations at day 14 after placement, the percentage in vivo vitreal level is between 25% and 65%, and more usually between 35% and 55%, relative to the level present at day 1 after placement. In some variations at day 14 after placement, the percentage in vivo vitreal level is greater than 25%, and more usually greater than 35%, relative to the level present at day 1 after placement.

In some variations at day 28 after placement, the percentage in vivo vitreal level is between 55% and 95%, and more usually between 85% and 85%, relative to the level present at day 1 after placement. In some variations at day 28 after placement, the percentage in vivo vitreal level is greater than 55%, and more usually greater than 65%, relative to the level present at day 1 after placement.

In some variations at day 75 after placement, the percentage in vivo vitreal level is between 5% and 30%, and more usually between 10% and 25%, relative to the level present at day 1 after placement. In some variations at day 75 after placement, the percentage in vivo vitreal level is greater than 5%, and more usually greater than 10%, relative to the level present at day 1 after placement.

In some variations at day 95 after placement, the percentage in vivo vitreal level is between 90% and 150%, and more usually between 100% and 130%, relative to the level present at day 1 after placement. In some variations at day 95 after placement, the percentage in vivo vitreal level is greater than 90%, and more usually greater than 100%, relative to the level present at day 1 after placement.

In some variations, the solid drug delivery systems described herein may have in vivo delivery to the retina choroid profiles with the following described characteristics, where the delivery profiles are for delivery of therapeutic agent in vivo after placement of the solid drug delivery system into the area between the sclera and the conjunctiva of a rabbit eye.

In some variations at day 14 after placement, the percentage in vivo retina choroid level is between 2% and 20%, and more usually between 5% and 10%, relative to the level present at day 1 after placement. In some variations at day 14 after placement, the percentage in vivo retina choroid level is greater than 2%, and more usually greater than 5%, relative to the level present at day 1 after placement.

In some variations at day 28 after placement, the percentage in vivo retina choroid level is between 5% and 45%, and more usually between 15% and 35%, relative to the level present at day 1 after placement. In some variations at day 28 after placement, the percentage in vivo retina choroid level is greater than 5%, and more usually greater than 15%, relative to the level present at day 1 after placement.

In some variations at day 75 after placement, the percentage in vivo retina choroid level is between 2% and 35%, and more usually between 10% and 20%, relative to the level present at day 1 after placement. In some variations at day 75 after placement, the percentage in vivo retina choroid level is greater than 2%, and more usually greater than 10%, relative to the level present at day 1 after placement.

In some variations at day 95 after placement, the percentage in vivo retina choroid level is between 1% and 15%, and more usually between 4% and 10%, relative to the level present at day 1 after placement. In some variations at day 95 after placement, the percentage in vivo vitreal level is greater than 1%, and more usually greater than 4%, relative to the level present at day 1 after placement.

In some variations, the solid drug delivery systems described herein may have in vivo clearance from the sclera profiles with the following described characteristics, where the clearance profiles are for delivery of therapeutic agent in vivo after placement of the solid drug delivery system into the area between the sclera and the conjunctiva of a rabbit eye.

In some variations at day 14 after placement, the percentage in vivo vitreal level is between 15% and 55%, and more usually between 25% and 45%, relative to the level present at day 1 after placement. In some variations at day 14 after placement, the percentage in vivo vitreal level is greater than 15%, and more usually greater than 55%, relative to the level present at day 1 after placement.

In some variations at day 28 after placement, the percentage in vivo vitreal level is between 75% and 115%, and more usually between 85% and 105%, relative to the level present at day 1 after placement. In some variations at day 28 after placement, the percentage in vivo vitreal level is greater than 75%, and more usually greater than 85%, relative to the level present at day 1 after placement.

In some variations at day 75 after placement, the percentage in vivo vitreal level is between 2% and 30%, and more usually between 5% and 15%, relative to the level present at day 1 after placement. In some variations at day 75 after placement, the percentage in vivo vitreal level is greater than 2%, and more usually greater than 5%, relative to the level present at day 1 after placement.

In some variations at day 95 after placement, the percentage in vivo vitreal level is between 0.5% and 10%, and more usually between 2% and 8%, relative to the level present at day 1 after placement. In some variations at day 95 after placement, the percentage in vivo vitreal level is greater than 0.5%, and more usually greater than 2%, relative to the level present at day 1 after placement.

The “average concentration” of a therapeutic agent is calculated by (1) performing an experiment including but not limited to placing a solid drug delivery system into the vitreous of a rabbit eye, (2) measuring the levels of the therapeutic agent in the rabbit eye using LCMS (liquid chromatography mass spectroscopy), and (3) taking the average of the levels obtained in the rabbit eyes. The average may be taken on any number higher than one. In some variations, the average is taken by adding the levels of therapeutic agent in 2 eyes of each of two rabbits and dividing by 4, where the solid drug delivery system was placed in each eye analyzed.

Described herein are solid drug delivery systems showing in vivo delivery or clearance profiles with one or more of the following characteristics. The delivery or clearance profiles are for clearance of the therapeutic agent in vivo after placement of the solid drug delivery system subconjunctivally in a rabbit eye. In some variations, the delivery or clearance profiles are for clearance of rapamycin in vivo after placement of the solid drug delivery system subconjunctivally or into the vitreous of a rabbit eye. The volume of the rabbit vitreous is approximately 30-40% of the volume of the human vitreous. The amount of therapeutic agent is measured using techniques as described in Example 2, but without limitation to the formulation and therapeutic agent described in Example 2.

In some variations, the therapeutic agents with the in vivo delivery or clearance profiles described herein include but are not limited to those described in the Therapeutic Agents section. In some variations the therapeutic agent is rapamycin. In some variations, the solid drug delivery systems described herein are used to deliver therapeutic agents in a concentration equivalent to rapamycin. The solid drug delivery systems described herein may comprise any therapeutic agent including but not limited to those in the Therapeutic Agents section, in a concentration equivalent to rapamycin, including but not limited to those concentrations described herein including in the examples.

The average concentration of a therapeutic agent over a period of time means for representative timepoints over the period of time the average concentration at each time point. For example, if the time period is 30 days, the average concentration may be measured at 5 day intervals: for the average concentration at day 5, the average of a number of measurements of concentration at day 5 would be calculated; for the average concentration at day 10, the average of a number of measurements of the concentration at day 10 would be calculated, etc.

In some variations, the solid drug delivery system when placed between the sclera and conjunctiva of a rabbit eye delivers therapeutic agent maintaining an average concentration of therapeutic agent in the vitreous of the rabbit eye of at least 0.01 pg/mL for at least 30, at least 60, at least 90, or at least 105 days after placement of the solid drug delivery system in the rabbit eye. In some variations, the solid drug delivery system when placed between the sclera and conjunctiva of a rabbit eye delivers therapeutic agent maintaining an average concentration of therapeutic agent in the vitreous of the rabbit eye of at least 0.001 ng/mL for at least 30, at least 60, at least 90, or at least 105 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the vitreous of the rabbit eye of at least 0.01 ng/mL for at least 30, at least 60, at least 90, or at least 105 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the vitreous of the rabbit eye of at least 0.1 ng/mL for at least 30, at least 60, at least 90, or at least 105 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the vitreous of the rabbit eye of at least 1 ng/mL for at least 30, at least 60, or at least 90 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the vitreous of the rabbit eye of at least 2.5 ng/mL for at least 30, at least 60, or at least 90 days after placement of the solid drug delivery system in the rabbit eyes.

In some variations, the solid drug delivery system when placed between the sclera and conjunctiva of a rabbit eye delivers therapeutic agent maintaining an average concentration of therapeutic agent in the retina choroid of the rabbit eye of at least 0.01 pg/mg for at least 30, at least 60, at least 90, or at least 105 days after placement of the solid drug delivery system in the rabbit eye. In some variations, the solid drug delivery system when placed between the sclera and conjunctiva of a rabbit eye delivers therapeutic agent maintaining an average concentration of therapeutic agent in the retina choroid of the rabbit eye of at least 0.1 pg/mg for at least 30, at least 60, at least 60, at least 90, or at least 105 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the retina choroid of the rabbit eye of at least 1 pg/mg for at least 30, at least 60, at least 90, or at least 105 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the retina choroid of the rabbit eye of at least 0.01 ng/mg for at least 30, at least 60, at least 90, or at least 105 days after placement of the solid drug delivery system in the rabbit eyes. In some variations, the solid drug delivery system when placed between the sclera and conjunctiva of a rabbit eye delivers therapeutic agent maintaining an average concentration of therapeutic agent in the retina choroid of the rabbit eye of at least 0.1 ng/mg for at least 30, at least 60, or at least 90 days after placement of the solid drug delivery system in the rabbit eyes.

In some variations, a solid drug delivery system described herein delivers a level of a therapeutic agent to the specified tissue that is approximately constant over a period of time. “Approximately constant,” as used herein, means that the average level does not vary by more than one order of magnitude over the extended period of time, i.e., the difference between the maximum and minimum is less than a 10-fold difference for measurements of the average concentration at times in the relevant period of time. In some variations, the therapeutic agent is rapamycin and the level of rapamycin is approximately constant over the specified period of time in the specified tissue.

In some variations, the solid drug delivery system when placed between the sclera and conjunctiva of a rabbit eye delivers therapeutic agent giving an average concentration of therapeutic agent in the vitreous of a rabbit eye that is approximately constant at a value greater than 0.001 ng/mL between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the vitreous of a rabbit eye that is approximately constant at a value greater than 0.01 ng/mL between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the vitreous of a rabbit eye that is approximately constant at a value greater than 0.1 ng/mL between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the vitreous of a rabbit eye that is approximately constant at a value of 0.75 ng/mL between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the vitreous of a rabbit eye that is approximately constant at a value of 1 ng/mL between day 14 to at least day 28, at least day 75, at least day 95, or at least day 107 after placement of the solid drug delivery system in the rabbit eye.

In some variations, the solid drug delivery system when placed between the sclera and conjunctiva of a rabbit eye delivers therapeutic agent giving an average concentration of therapeutic agent in the retina choroid of the rabbit eye of at least 0.001 ng/mg between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the retina choroid of the rabbit eye of at least 0.005 ng/mg between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the retina choroid of the rabbit eye of at least 0.01 between day 14 to at least day 28, at least day 75, at least day 95, or at least day 107 after placement of the solid drug delivery system in the rabbit eye. In some variations, the solid drug delivery system when placed between the sclera and conjunctiva of a rabbit eye delivers therapeutic agent giving an average concentration of therapeutic agent in the retina choroid of the rabbit eye of at least 0.03 between day 14 to at least day 28, at least day 75, at least day 95, or at least day 107 after placement of the solid drug delivery system in the rabbit eye.

In some variations, the solid drug delivery system when placed between the sclera and conjunctiva of a rabbit eye delivers therapeutic agent giving an average concentration of therapeutic agent in the sclera of the rabbit eye of at least 0.001 ng/mg between day 42 to at least day 63, or at least day 91 after placement of the solid drug delivery system in the rabbit eye. In some variations, the solid drug delivery system when placed between the sclera and conjunctiva of a rabbit eye delivers therapeutic agent giving an average concentration of therapeutic agent in the sclera of the rabbit eye of at least 0.005 ng/mg between day 42 to at least day 63, or at least day 91 after placement of the solid drug delivery system in the rabbit eye. In some variations, the solid drug delivery system when placed between the sclera and conjunctiva of a rabbit eye delivers therapeutic agent giving an average concentration of therapeutic agent in the sclera of the rabbit eye of at least 0.01 ng/mg between day 42 to at least day 63, or at least day 91 after placement of the solid drug delivery system in the rabbit eye. In some variations, the solid drug delivery system when placed between the sclera and conjunctiva of a rabbit eye delivers therapeutic agent giving an average concentration of therapeutic agent in the sclera of the rabbit eye of at least 0.03 ng/mg between day 42 to at least day 63, or at least day 91 after placement of the solid drug delivery system in the rabbit eye. In some variations, the solid drug delivery system when placed between the sclera and conjunctiva of a rabbit eye delivers therapeutic agent giving an average concentration of therapeutic agent in the sclera of the rabbit eye of at least 0.1 ng/mg between day 42 to at least day 63, or at least day 91 after placement of the solid drug delivery system in the rabbit eye. In some variations, the solid drug delivery system when placed between the sclera and conjunctiva of a rabbit eye delivers therapeutic agent giving an average concentration of therapeutic agent in the sclera of the rabbit eye of at least 1.0 ng/mg between day 42 to at least day 63, or at least day 91 after placement of the solid drug delivery system in the rabbit eye.

In some variations, the solid drug delivery system when placed between the sclera and conjunctiva of a rabbit eye delivers a therapeutic agent to give an average concentration of the therapeutic agent in the vitreous of the rabbit eye of between 0.001 and 15.0 ng/ml for at least 14, at least 28, at least 75, at least 95, or at least 107 days after administration of the solid drug delivery system to the rabbit eye. In some variations, the solid drug delivery system when placed between the sclera and conjunctiva of a rabbit eye delivers a therapeutic agent to give an average concentration of the therapeutic agent in the vitreous of the rabbit eye of between 0.01 and 10.0 ng/ml for at least 14, at least 28, at least 75, at least 95, or at least 107 days after administration of the solid drug delivery system to the rabbit eye. In some variations, the solid drug delivery system when placed between the sclera and conjunctiva of a rabbit eye delivers a therapeutic agent to give an average concentration of the therapeutic agent in the vitreous of the rabbit eye of between 0.1 and 10.0 ng/ml for at least 14, at least 28, at least 75, at least 95, or at least 107 days after administration of the solid drug delivery system to the rabbit eye.

In some variations, the solid drug delivery system when placed between the sclera and conjunctiva of a rabbit eye delivers a therapeutic agent to give an average concentration of the therapeutic agent in the retina choroid of the rabbit eye of between 0.001 and 5.0 ng/mg for at least 14, at least 28, at least 75, at least 95, or at least 107 days after administration of the solid drug delivery system to the rabbit eye. In some variations, the solid drug delivery system when placed between the sclera and conjunctiva of a rabbit eye delivers a therapeutic agent to give an average concentration of the therapeutic agent in the retina choroid of the rabbit eye of between 0.001 and 1.25 ng/mg for at least 14, at least 28, at least 75, at least 95, or at least 107 days after administration of the solid drug delivery system to the rabbit eye. In some variations, the solid drug delivery system when placed between the sclera and conjunctiva of a rabbit eye delivers a therapeutic agent to give an average concentration of the therapeutic agent in the retina choroid of the rabbit eye of between 0.01 and 5.0 ng/mg for at least 14, at least 28, at least 75, at least 95, or at least 107 days after administration of the solid drug delivery system to the rabbit eye.

In some variations, the solid drug delivery system when placed between the sclera and conjunctiva of a rabbit eye delivers a therapeutic agent to give an average concentration of the therapeutic agent in the sclera of the rabbit eye of between 0.001 and 10.0 ng/mg for at least 14, at least 28, at least 75, at least 95, or at least 107 days after administration of the solid drug delivery system to the rabbit eye. In some variations, the solid drug delivery system when placed between the sclera and conjunctiva of a rabbit eye delivers a therapeutic agent to give an average concentration of the therapeutic agent in the sclera of the rabbit eye of between 0.01 and 10.0 ng/mg for at least 14, at least 28, at least 75, at least 95, or at least 107 days after administration of the solid drug delivery system to the rabbit eye. In some variations, the solid drug delivery system when placed between the sclera and conjunctiva of a rabbit eye delivers a therapeutic agent to give an average concentration of the therapeutic agent in the sclera of the rabbit eye of between 0.1 and 200.0 ng/mg for at least 14, at least 42, at least 63, or at least 91 days after administration of the solid drug delivery system to the rabbit eye.

Therapeutic Agents

Most generally, any compounds and compositions currently known or yet to be discovered that are useful in treating, preventing, inhibiting, delaying the onset of, or causing the regression of the diseases and conditions described herein may be therapeutic agents for use in the solid drug delivery systems and methods described herein.

Therapeutic agents that may be used include compounds that act by binding members of the immunophilin family of cellular proteins. Such compounds are known as “immunophilin binding compounds.” Immunophilin binding compounds include but are not limited to the “limus” family of compounds. Examples of compounds that may be used include but are not limited to cyclophilins, sirolimus (rapamycin) and its water soluble analog SDZ-RAD (Novartis), TAFA-93 (Isotechnika), tacrolimus, everolimus, RAD-001 (Novartis), pimecrolimus, temsirolimus, CCI-779 (Wyeth), AP23841 (Ariad), AP23573 (Ariad), and ABT-578 (Abbott Laboratories). Limus compound analogs and derivatives that may be used include but are not limited to the compounds described in U.S. Pat. Nos. 5,527,907; 6,376,517; and 6,329,386 and U.S. patent application Ser. No. 09/950,307, each of which is incorporated herein by reference in their entirety. Therapeutic agents also include analogs, prodrugs, salts and esters of limus compounds.

The terms rapamycin, rapa, and sirolimus are used interchangeably herein.

Other rapamycin derivatives that may be used include, without limitation, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, mono- and di-ester derivatives of rapamycin, 27-oximes of rapamycin; 42-oxo analog of rapamycin; bicyclic rapamycins; rapamycin dimers; silyl ethers of rapamycin; rapamycin arylsulfonates and sulfamates, mono-esters and di-esters at positions 31 and 42, 30-demethoxy rapamycin, and other derivatives described in Vezina et al., “Rapamycin (AY-22,989), A New Antifungal Antibiotic. I. Taxonomy Of The Producing Streptomycete And Isolation Of The Active Principle” J. Antibiot. (Tokyo) 28:721-726 (1975); Sehgal et al., “Rapamycin (AY-22,989), A New Antifungal Antibiotic. II. Fermentation, Isolation And Characterization” J. Antibiot. (Tokyo) 28:727-732 (1975); Sehgal et al., “Demethoxyrapamycin (AY-24,668), A New Antifungal Antibiotic” J. Antibiot. (Tokyo) 36:351-354 (1983); and Paiva et al., “Incorporation Of Acetate, Propionate, And Methionine Into Rapamycin By Streptomycetes hygroscopicus” J Nat Prod 54:167-177 (1991), WO 92/05179, EP 467606, Caufield et al., “Hydrogenated Rapamycin Derivatives” U.S. Pat. No. 5,023,262; Kao et al., “Bicyclic Rapamycins” U.S. Pat. No. 5,120,725; Kao et al., “Rapamycin Dimers” U.S. Pat. No. 5,120,727; Failli et al., “Silyl Ethers Of Rapamycin” U.S. Pat. No. 5,120,842; Failli et al., “Rapamycin 42-Sulfonates And 42-(N-carboalkoxy) Sulfamates Useful As Immunosuppressive Agents” U.S. Pat. No. 5,177,203; Nicolaou et al., “Total Synthesis Of Rapamycin” J. Am. Chem. Soc. 115: 4419-4420 (1993); Romo et al, “Total Synthesis Of (−) Rapamycin Using An Evans-Tishchenko Fragment Coupling” J. Am. Chem. Soc. 115:7906-7907 (1993); and Hayward et al, “Total Synthesis Of Rapamycin Via A Novel Titanium-Mediated Aldol Macrocyclization Reaction” J. Am. Chem. Soc., 115:9345-9346 (1993), each of which is incorporated herein by reference in its entirety.

The limus family of compounds may be used in the solid drug delivery systems and methods for the treatment, prevention, inhibition, delaying the onset of, or causing the regression of angiogenesis-mediated diseases and conditions of the eye, including choroidal neovascularization. The limus family of compounds may be used to prevent, treat, inhibit, delay the onset of, or cause regression of AMD, including wet AMD. Rapamycin and rapamycin derivatives and analogs may be used to prevent, treat, inhibit, delay the onset of, or cause regression of angiogenesis-mediated diseases and conditions of the eye, including choroidal neovascularization. Rapamycin may be used to prevent, treat, inhibit, delay the onset of, or cause regression of AMD, including wet AMD. In some variations, a member of the limus family of compounds or rapamycin is used to treat wet AMD or angiogenesis-mediated diseases and conditions of the eye including choroidal neovascularization.

Other therapeutic agents that may be used include those disclosed in the following patents and publications, the contents of each of which is incorporated herein by reference in its entirety: PCT publication WO 2004/027027, published Apr. 1, 2004, titled Method of inhibiting choroidal neovascularization, assigned to Trustees of the University of Pennsylvania; U.S. Pat. No. 5,387,589, issued Feb. 7, 1995, titled Method of Treating Ocular Inflammation, with inventor Prassad Kulkarni, assigned to University of Louisville Research Foundation; U.S. Pat. No. 6,376,517, issued Apr. 23, 2003, titled Pipecolic acid derivatives for vision and memory disorders, assigned to GPI NIL Holdings, Inc; PCT publication WO 2004/028477, published Apr. 8, 2004, titled Method subretinal administration of therapeutics including steroids: method for localizing pharmadynamic action at the choroid and retina; and related methods for treatment and or prevention of retinal diseases, assigned to Innorx, Inc; U.S. Pat. No. 6,416,777, issued Jul. 9, 2002, titled Ophthalmic drug delivery device, assigned to Alcon Universal Ltd; U.S. Pat. No. 6,713,081, issued Mar. 30, 2004, titled Ocular therapeutic agent delivery device and methods for making and using such devices, assigned to Department of Health and Human Services; U.S. Pat. No. 5,100,899, issued Mar. 31, 1992, titled Methods of inhibiting transplant rejection in mammals using rapamycin and derivatives and prodrugs thereof.

Other therapeutic agents that may be used include pyrrolidine, dithiocarbamate (NFκB inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibuzumab (Lucentis™) and other antibodies directed to the same target; pegaptanib (Macugen™); vitronectin receptor antagonists, such as cyclic peptide antagonists of vitronectin receptor-type integrins; α-v/β-3 integrin antagonists; α-v/β-1 integrin antagonists; thiazolidinediones such as rosiglitazone or troglitazone; interferon, including γ-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; apoptosis inhibiting agents; Visudyne™, snET2 and other photo sensitizers, which may be used with photodynamic therapy (PDT); inhibitors of hepatocyte growth factor (antibodies to the growth factor or its receptors, small molecular inhibitors of the c-met tyrosine kinase, truncated versions of HGF e.g. NK4).

Other therapeutic agents that may be used include anti-inflammatory agents, including, but not limited to nonsteroidal anti-inflammatory agents and steroidal anti-inflammatory agents. In some variations, active agents that may be used in the solid drug delivery systems are ace-inhibitors, endogenous cytokines, agents that influence basement membrane, agents that influence the growth of endothelial cells, adrenergic agonists or blockers, cholinergic agonists or blockers, aldose reductase inhibitors, analgesics, anesthetics, antiallergics, antibacterials, antihypertensives, pressors, antiprotozoal agents, antiviral agents, antifungal agents, anti-infective agents, antitumor agents, antimetabolites, and antiangiogenic agents.

Steroidal therapeutic agents that may be used include but are not limited to 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, and any of their derivatives.

In some variations, cortisone, dexamethasone, fluocinolone, hydrocortisone, methylprednisolone, prednisolone, prednisone, and triamcinolone, or their derivatives, may be used. The solid drug delivery system may include a combination of two or more steroidal therapeutic agents.

In one nonlimiting example, the steroidal therapeutic agents may constitute from 0.05% to 50% by weight of the solid drug delivery system. In another nonlimiting example, the steroid constitutes from 0.05% to 10%, between 10% to 20%; between 30% to 40%; or between 40% to 50% by weight of the solid drug delivery system.

Other nonlimiting examples of therapeutic agents that may be used include but are not limited to anaesthetics, analgesics, cell transport/mobility impending agents such as colchicines, vincristine, cytochalasin B and related compounds; carbonic anhydrase inhibitors such as acetazolamide, methazolamide, dichlorphenamide, diamox and neuroprotectants such as nimodipine and related compounds; antibiotics such as tetracycline, chlortetracycline, bacitracin, neomycin, polymyxin, gramicidin, cephalexin, oxytetracycline, chloramphenicol, rifampicin, ciprofloxacin, aminosides, gentamycin, erythromycin and penicillin, quinolone, ceftazidime, vancomycine imipeneme; antifungals such as amphotericin B, fluconazole, ketoconazole and miconazole; antibacterials such as sulfonamides, sulfadiazine, sulfacetamide, sulfamethizole and sulfisoxazole, nitrofurazone and sodium propionate; antivirals, such as idoxuridine, trifluorothymidine, trifluorouridine, acyclovir, ganciclovir, cidofovir, interferon, DDI, AZT, foscamet, vidarabine, irbavirin, protease inhibitors and anti-cytomegalovirus agents; antiallergenics such as sodium cromoglycate, antazoline, methapyriline, chlorpheniramine, cetirizine, pyrilamine and prophenpyridamine; synthetic gluocorticoids and mineralocorticoids and more generally hormones forms derivating from the cholesterol metabolism (DHEA, progesterone, estrogens); non-steroidal anti-inflammatories such as salicylate, indomethacin, ibuprofen, diclofenac, flurbiprofen, piroxicam and COX2 inhibitors; antineoplastics such as carmustine, cisplatin, fluorouracil; adriamycin, asparaginase, azacitidine, azathioprine, bleomycin, busulfan, carboplatin, carmustine, chlorambucil, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, florxymesterone, flutamide, goserelin, hydroxyurea, ifosfamide, leuprolide, levamisole, limustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil mustard, vinblastine, vincristine and vindesine; immunological drugs such as vaccines and immune stimulants; insulin, calcitonin, parathyroid hormone and peptide and vasopressin hypothalamus releasing factor; beta adrenergic blockers such as timolol, levobunolol and betaxolol; cytokines, interleukines and growth factors epidermal growth factor, fibroblast growth factor, platelet derived growth factor, transforming growth factor beta, ciliary neurotrophic growth factor, glial derived neurotrophic factor, NGF, EPO, PLGF, brain nerve growth factor (BNGF), vascular endothelial growth factor (VEGF) and monoclonal antibodies or fragments thereof directed against such growth factors; anti-inflammatories such as hydrocortisone, dexamethasone, fluocinolone, prednisone, prednisolone, methylprednisolone, fluorometholone, betamethasone and triamcinolone; decongestants such as phenylephrine, naphazoline and tetrahydrazoline; miotics and anti-cholinesterases such as pilocarpine, carbachol, di-isopropyl fluorophosphate, phospholine iodine and demecarium bromide; mydriatics such as atropine sulphate, cyclopentolate, homatropine, scopolamine, tropicamide, eucatropine; sympathomimetics such as epinephrine and vasoconstrictors and vasodilators, anticlotting agents such as heparin, antifibrinogen, fibrinolysin, anticlotting activase, antidiabetic agents include acetohexamide, chlorpropamide, glipizide, glyburide, tolazamide, tolbutamide, insulin and aldose reductase inhibitors, hormones, peptides, nucleic acids, saccharides, lipids, glycolipids, glycoproteins and other macromolecules include endocrine hormones such as pituitary, insulin, insulin-related growth factor, thyroid, growth hormones; heat shock proteins; immunological response modifiers such as muramyl dipeptide, cyclosporins, interferons (including alpha-, beta- and gamma-interferons), interleukin-2, cytokines, FK506 (an epoxy-pyrido-oxaazcyclotricosine-tetrone, also known as Tacrolimus), tumor necrosis factor, pentostatin, thymopentin, transforming factor beta2, erythropoetin; antineogenesis proteins (e.g. anti VEGF, interferons), antibodies (monoclonal, polyclonal, humanized, etc.) or antibodies fragments, oligoaptamers, aptamers and gene fragments (oligonucleotides, plasmids, ribozymes, small interference RNA (SiRNA), nucleic acid fragments, peptides), immunomodulators such as endoxan, thalidomide, tamoxifene; antithrombolytic and vasodilator agents such as rtPA, urokinase, plasmin; nitric oxide donors, nucleic acids, dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur) fluocinolone, triaminolone, anecortave acetate, fluorometholone, medrysone, and prednislone. In some variations the immunosuppressive agent is dexamethasone. In some variations the immunosuppressive agent is cyclosporin A.

In some variations the formulation comprises a combination of one or more therapeutic agents.

Other nonlimiting examples of therapeutic agents that may be used in the formulations described herein include antibacterial antibiotics, aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), P-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefminox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), ritipenem, lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin); synthetic antibacterials, 2.4-Diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, n2-formylsulfisomidine, n4-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n4-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene-citrate, methenamine hippurate, methenamine mandelate, methenamine sulfosalicylate, nitroxoline, taurolidine, xibomol), antifungal antibiotics, polyenes (e.g., amphotericin b, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyrrolnitrin, siccanin, tubercidin, viridin, synthetic antifungals, allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate, antineoplastics, antibiotics and analogs (e.g., aclacinomycins, actinomycin f1, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), antimetabolites (e.g. folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tagafur), antiinflammatory agents, steroidal antiinflammatory agents, acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide, non-steroidal antiinflammatory agents, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lomoxicam, piroxicam, tenoxicam), ε-acetamidocaproic acid, s-adenosylmethionine, 3-amino-4-hydroxybutyric acid, amixetrine, bendazac, benzydamine, a-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

The therapeutic agents may also be used in combination with other therapeutic agents and therapies, including but not limited to agents and therapies useful for the treatment, prevention, inhibition, delaying onset of, or causing regression of angiogenesis or neovascularization, particularly CNV. In some variations the additional agent or therapy is used to treat regression of angiogenesis or neovascularization, particularly CNV. Non-limiting examples of such additional agents and therapies include pyrrolidine, dithiocarbamate (NFκB inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibuzumab (Lucentis™) and other antibodies directed to the same target; pegaptanib (Macugen™); vitronectin receptor antagonists, such as cyclic peptide antagonists of vitronectin receptor-type integrins; α-v/β-3 integrin antagonists; α-v/β-1 integrin antagonists; thiazolidinediones such as rosiglitazone or troglitazone; interferon, including γ-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; inhibitors of hepatocyte growth factor (antibodies to the growth factor or its receptors, small molecular inhibitors of the c-met tyrosine kinase, truncated versions of HGF e.g. NK4); apoptosis inhibiting agents; Visudyne™, snET2 and other photo sensitizers with photodynamic therapy (PDT); and laser photocoagulation.

Diseases and Conditions that May be Treated, Prevented, Inhibited, Onset Delayed, or Regression Caused

Herein are described diseases and conditions that may be treated, prevented, inhibited, onset delayed, or regression caused using the therapeutic agents, solid drug delivery systems and methods described herein. In some variations, the diseases or conditions are treated using one or more of a solid drug delivery system comprising a therapeutic agent or a method described herein. Unless the context indicates otherwise, it is envisioned that the subjects on whom all of the methods of treatment may be performed include, but are not limited to, human subjects.

Generally, any disease or condition of the eye susceptible to treatment, prevention, inhibition, delaying the onset of, or causing the regression of using the therapeutic agents and the solid drug delivery systems and methods described herein may be treated, prevented, inhibited, onset delayed, or regression caused treated or prevented. Examples of diseases or conditions of the eye include, but are not limited to, diseases or conditions associated with neovascularization including retinal and/or choroidal neovascularization.

Diseases or conditions associated with retinal and/or choroidal neovascularization that can be treated, prevented inhibited, have onset delayed, or be caused to regress using the solid drug delivery systems and methods described herein include, but are not limited to, diabetic retinopathy, macular degeneration, wet and dry AMD, retinopathy of prematurity (retrolental fibroplasia), infections causing a retinitis or choroiditis, presumed ocular histoplasmosis, myopic degeneration, angioid streaks, and ocular trauma. Other non-limiting examples of diseases and conditions of the eye that may be treated, prevented inhibited, have onset delayed, or be caused to regress using the solid drug delivery systems and methods described herein include, but are not limited to, pseudoxanthoma elasticum, vein occlusion, artery occlusion, carotid obstructive disease, Sickle Cell anemia, Eales disease, myopia, chronic retinal detachment, hyperviscosity syndromes, toxoplasmosis, trauma, polypoidal choroidal vasculopathy, post-laser complications, complications of idiopathic central serous chorioretinopathy, complications of choroidal inflammatory conditions, rubeosis, diseases associated with rubeosis (neovascularization of the angle), neovascular glaucoma, uveitis and chronic uveitis, macular edema, proliferative retinopathies and diseases or conditions caused by the abnormal proliferation of fibrovascular or fibrous tissue, including all forms of proliferative vitreoretinopathy (including post-operative proliferative vitreoretinopathy), whether or not associated with diabetes.

When used to treat, prevent, inhibit, delay the onset of, or cause regression of uveitis, the solid drug delivery systems described herein may be placed in the subject by a variety or routes as is known in the art, including but not limited to by ocular or oral administration. Other methods of placement are known and are routine in the art. Some examples thereof are listed in the herein.

One disease that may be treated, prevented inhibited, have onset delayed, or be caused to regress using the solid drug delivery systems and methods described herein is the wet form of AMD. The wet form of AMD is characterized by blood vessels growing from their normal location in the choroid into an undesirable position under the retina. Leakage and bleeding from these new blood vessels results in vision loss and possibly blindness.

The dry form of AMD is associated with the retinal pigment epithelium or RPE degenerating and leading to photoreceptor cell death, and the formation of yellow deposits called drusen under the retina. The solid drug delivery systems and methods described herein may also be used to prevent or slow the transition from the dry form of AMD to the wet form of AMD.

“Macular degeneration” is characterized by the excessive buildup of fibrous deposits in the macula and retina and the atrophy of the retinal pigment epithelium. As used herein, an eye “afflicted” with macular degeneration is understood to mean that the eye exhibits at least one detectable physical characteristic associated with the disease of macular degeneration. The administration of rapamycin appears to limit excessive angiogenesis, such as choroidal neovascularization in age-related macular degeneration (AMD), which may occur without such treatment. As used herein, the term “angiogenesis” means the generation of new blood vessels (“neovascularization”) into a tissue or organ. An “angiogenesis-mediated disease or condition” of the eye or retina is one in which new blood vessels are generated in a pathogenic manner in the eye or retina, resulting in loss of vision or other problem, e.g., choroidal neovascularization associated with AMD.

The solid drug delivery systems described herein, including but not limited to rapamycin-containing solid drug delivery systems, may also be used to treat, prevent, inhibit, delay the onset of, or cause regression of various immune-related diseases and conditions, including but not limited to organ transplant rejection in a host, graft vs. host disease, autoimmune diseases, diseases of inflammation, hyperproliferative vascular disorders, solid tumors, and fungal infections. The solid drug delivery systems described herein, including but not limited to rapamycin-containing solid drug delivery systems, may be used as immunosuppressants. The solid drug delivery systems described herein, including but not limited to rapamycin-containing solid drug delivery systems, may be used to treat, prevent, inhibit, or delay the onset of rejection of transplanted organs or tissues including but not limited to transplanted heart, liver, kidney, spleen, lung, small bowel, pancreas, and bone marrow. When used to treat, prevent, inhibit, delay the onset of, or cause regression of immune-related diseases, including but not limited to transplant rejection, the solid drug delivery systems described herein may be placed in the subject by a variety or routes as is known in the art, including but not limited to by oral administration.

In some variations, the solid drug delivery systems described herein are used to prevent or delay onset of a disease or condition of the eye where the subject, including but not limited to a human subject, is at heightened risk of developing the disease or condition of the eye. A subject with a heightened risk of developing a disease or condition is a subject with one or more indications that the disease or condition is likely to develop in the particular subject.

In some variations the subject with a heightened risk of developing wet AMD is a subject with dry AMD in at least one eye. In some variations the subject with a heightened risk of developing wet AMD in a fellow eye is a subject with wet AMD in the other eye. In some variations, the solid drug delivery systems described herein are used to prevent or delay onset of CNV in a subject at heightened risk of developing CNV, including but not limited to prevention or delaying onset of CNV in the fellow eye of a subject, including but not limited to a human subject with AMD in one eye. In some variations, the solid drug delivery systems described herein are used to prevent or delay onset of CNV in the fellow eye of a subject with wet AMD in one eye.

In some variations, the solid drug delivery systems comprise a limus compound, including but not limited to rapamycin.

In some variations the solid drug delivery systems are administered periocularly, including without limitation subconjunctivally, to a human subject with vision of 20/40 or better. In some variations, the solid drug delivery systems are administered periocularly, including without limitation subconjunctivally or transsclerally, to the eye of a human subject where the eye to which the formulation is administered has vision of 20/40 or better.

In some variations, the solid drug delivery systems described herein are used to treat, prevent, or delay onset of AMD. In some variations, the solid drug delivery systems described herein are used to treat, prevent, or delay onset of dry AMD. In some variations, subjects including but not limited to human subjects with non-central geographic atrophy are administered a solid drug delivery system described herein to treat, prevent, or delay onset of central geographic atrophy. In some variations, the solid drug delivery systems comprise a limus compound, including but not limited to rapamycin. In some variations the solid drug delivery systems are administered periocularly, including without limitation subconjunctivally or transsclerally, to a human subject with vision of 20/40 or better. In some variations, the solid drug delivery systems described herein are administered and the subject, including but not limited to a human subject is also treated with a second therapy for treating the disease or disorder. In some variations, the solid drug delivery systems described herein are used to treat, prevent, or delay onset of wet or dry AMD and the subject, including but not limited to a human subject is also treated with laser therapy such as photodynamic laser therapy, either before, during, or after treatment with the formulations or pharmaceutical formulations described herein.

In some variations, the solid drug delivery systems described herein are used to treat one or more of uveitis, allergic conjunctivitis, macular edema, glaucoma, or dry eye.

In some variations, a solid drug delivery system comprises a limus compound such as rapamycin, and is administered to treat, prevent, or delay onset of dry eye. In some variations, a solid drug delivery system comprises a limus compound such as rapamycin, and is administered to treat, prevent, or delay onset of allergic conjunctivitis.

In some variations, the methods or solid drug delivery systems described herein are used to treat retinitis pigmentosa. In some variations, the solid drug delivery systems described herein comprise a limus compound such as rapamycin, and are used to treat, prevent, or delay onset of retinitis pigmentosa. In some variations, the solid drug delivery systems described herein have a neuroprotective effect and are used to treat retinitis pigmentosa.

In some variations, the solid drug delivery systems described herein are used to treat one or more of central retinal vein occlusive diseases (CRVO), branch retinal venous occlusion (BRVO), retinal vascular diseases and conditions, macular edema, diabetic macular edema, iris neovascularization, diabetic retinopathy, or corneal graft rejection. In some variations, a solid drug delivery system comprises a limus compound such as rapamycin, and is administered to treat, prevent, or delay onset of one or more of these diseases or conditions. In some variations the solid drug delivery systems are administered subconjunctivally to an eye with vision of 20/40 or better.

Routes of administration are described elsewhere herein.

Other diseases and conditions that may be treated, prevented, inhibited, have the onset delayed, or be caused to regress using the methods described herein include those disclosed in the following patents and publications, the contents of each of which is incorporated herein in its entirety: PCT publication WO 2004/027027, published Apr. 1, 2004, titled Method of inhibiting choroidal neovascularization, assigned to Trustees of the University of Pennsylvania; U.S. Pat. No. 5,387,589, issued Feb. 7, 1995, titled Method of Treating Ocular Inflammation, with inventor Prassad Kulkami, assigned to University of Louisville Research Foundation; U.S. Pat. No. 6,376,517, issued Apr. 23, 2003, titled Pipecolic acid derivatives for vision and memory disorders, assigned to GPI NIL Holdings, Inc; PCT publication WO 2004/028477, published Apr. 8, 2004, titled Method subretinal administration of therapeutics including steroids: method for localizing pharmadynamic action at the choroid and retina; and related methods for treatment and or prevention of retinal diseases, assigned to Innorx, Inc; U.S. Pat. No. 6,416,777, issued Jul. 9, 2002, titled Ophthalmic drug delivery device, assigned to Alcon Universal Ltd; U.S. Pat. No. 6,713,081, issued Mar. 30, 2004, titled Ocular therapeutic agent delivery device and methods for making and using such devices, assigned to Department of Health and Human Services; and U.S. Pat. No. 5,536,729, issued Jul. 16, 1996, titled Rapamycin Formulations for Oral Administration, assigned to American Home Products Corp., and U.S. Pat. App. No. 60/503,840 and Ser. No. 10/945,682.

When a certain amount of a solid drug delivery system is administered, it is understood that there is some imprecision in the accuracy of various devices that may be used to administer the solid drug delivery system. Where a certain amount is specified, it is understood that this is the target amount.

When the therapeutic agent is rapamycin, the solid drug delivery system may be used to maintain an amount of rapamycin in the vitreous effective to treat wet AMD. In one nonlimiting example, it is believed that a solid drug delivery system delivering rapamycin to maintain a concentration of rapamycin of 10 pg/ml to 2 μg/ml in the vitreous over a period of time may be used for the treatment of wet AMD. In another nonlimiting example, it is believed that a delivery system delivering rapamycin to maintain a concentration of rapamycin of 0.01 pg/mg to 10 ng/mg in the retina choroid over a period of time may be used for treatment of wet AMD. Other therapeutically effective amounts of therapeutic agent are also possible, and can be readily determined by one of skill in the art given the teachings herein.

When the therapeutic agent is rapamycin, the solid drug delivery systems described herein may be used to deliver a dose of rapamycin to a subject, including but not limited to a human subject or to the eye of a subject. In one nonlimiting example, it is believed that a solid drug delivery system containing a dose of 20 μg to 4 mg may be used for the treatment of wet AMD.

The amount of therapeutic agent component delivered may also be represented as a concentration equivalent to rapamycin. As used herein, “a concentration equivalent to rapamycin” refers to a concentration of a therapeutic agent that will have approximately the same efficacy in vivo as a particular dose of rapamycin for treating, preventing, delaying, or inhibiting a disease or condition, including but not limited to the diseases and conditions described herein. As a nonlimiting example, if a therapeutic agent is found to be approximately 25-fold less potent or efficacious than rapamycin in the treatment of wet AMD, a concentration of 25 ng/ml of the therapeutic agent would be equivalent to a 1 ng/ml concentration of rapamycin when used for the treatment of wet AMD.

Those of skill in the art, based on the teachings herein can determine what amount or concentration of a given therapeutic agent is equivalent to an amount or concentration of rapamycin by, for example, administering the therapeutic agent at various amounts or concentrations to a disease model system, such as an in vivo or in vivo model system, and comparing the results in the model system relative to the results of various amounts or concentrations of rapamycin. Those of skill in the art, based on the teachings herein can also determine what amount or concentration of a given therapeutic agent is equivalent to an amount or concentration of rapamycin by reviewing the scientific literature for experiments performed comparing rapamycin to other therapeutic agents. It is understood that even the same therapeutic agent may have a different equivalent level of rapamycin when, for example, a different disease or disorder is being evaluated, or a different type of formulation is used. Nonlimiting examples of scientific references with comparative studies of rapamycin and other therapeutic agents on ocular disease are Ohia et al., Effects of steroids and immunosuppressive drugs on endotoxin-uveitis in rabbits, J. Ocul. Pharmacol. 8(4):295-307 (1992); Kulkarni, Steroidal and nonsteroidal drugs in endotoxin-induced uveitis, J. Ocul. Pharmacol. 10(1):329-34 (1994); Hafizi et al., Differential effects of rapamycin, cyclosporine A, and FK506 on human coronary artery smooth muscle cell proliferation and signaling, Vascul Pharmacol. 41(4-5):167-76 (2004); and U.S. 2005/0187241.

For example, in a model for wet AMD, if a therapeutic agent is found to be approximately 10-fold less potent or efficacious than rapamycin in the treatment of wet AMD, a concentration of 10 ng/ml of the therapeutic agent would be equivalent to a 1 ng/ml concentration of rapamycin. Or if a therapeutic agent is found to be approximately 10-fold less potent or efficacious than rapamycin in the treatment of wet AMD, a 10-fold amount of the therapeutic agent would be administered relative to the amount of rapamycin.

Methods of Preparing Solid Drug Delivery Systems

Various methods as are known to those versed in the technology may be used to prepare the solid drug delivery systems described herein. In one method described herein, a solid drug delivery system may be made by mixing the therapeutic agent with an excipient including but not limited to a solvent, adding other excipient(s) as desired, and shaping the resulting solid drug delivery system. In some variations, after the solid drug delivery system was prepared the solvent was substantially absent from the solid drug delivery system. In some variations the solvent was evaporated using known methods of drying.

Various methods of shaping the solid drug delivery system may be used. In some variations, the solid drug delivery system is cast as a film on a sheet of released coated paper or polyester film, using a die (knife-over-roll). The resultant solid drug delivery system may subsequently be die-cut into a size and shape.

In some variations, the therapeutic agent and the excipient or excipients are blended by thermal melting. The mix may be sent through an extruder and a die to obtain a solid drug delivery system. The mix can also be injection-molded to a specific shape and size wafer.

In some variations, a solid drug delivery system described herein is made stable by a method described in U.S. 60/772,018, filed Feb. 9, 2006 with attorney docket number 57796-30010.00, titled STABLE FORMULATIONS, AND METHODS OF THEIR PREPARATION AND USE, which is incorporated herein by reference in its entirety for all purposes. In some variations, a solid drug delivery system described herein is prepared or is preparable by a method described in U.S. 60/772,018, filed Feb. 9, 2006 with attorney docket number 57796-30010.00, titled STABLE FORMULATIONS, AND METHODS OF THEIR PREPARATION AND USE.

Methods of Treatment

Unless the context clearly indicates otherwise, any of the therapeutic agents described herein may be used in a method for treating, preventing, inhibiting, delaying on set of, or causing the regression of any of the diseases and conditions described herein.

In some variations any one or more of the solid drug delivery systems described herein are used to deliver one or more therapeutic agents described herein via a method described herein. Generally, the therapeutic agent may be formulated in any solid drug delivery system capable of delivery of a therapeutically effective amount of the therapeutic agent to a subject or to the subject for the required treatment period. In some variations the required treatment period is met by a single administration of a sustained release solid drug delivery system that is predicted to deliver an effective amout of the therapeutic agent for the predicted duration period of the disease or condition. In some variations the required treatment period is met by multiple administrations of a solid drug delivery system, including but not limited to a sustained release formulation. In some variations the multiple administrations are at different times, at the same time in different places, or a combination thereof.

As used herein, to “inhibit” a disease or condition by administration of a therapeutic agent means that the progress of at least one detectable physical characteristic or symptom of the disease or condition is slowed or stopped following administration of the therapeutic agent as compared to the progress of the disease or condition without administration of the therapeutic agent.

As used herein, to “prevent” a disease or condition by administration of a therapeutic agent means that the detectable physical characteristics or symptom of the disease or condition do not develop following administration of the therapeutic agent.

As used herein, to “delay onset of” a disease or condition by administration of a therapeutic agent means that at least one detectable physical characteristic or symptom of the disease or condition develops later in time following administration of the therapeutic agent as compared to the progress of the disease or condition without administration of the therapeutic agent.

As used herein, to “treat” a disease or condition by administration of a therapeutic agent means that the progress of at least one detectable physical characteristic or symptom of the disease or condition is slowed, stopped, or reversed following administration of the therapeutic agent as compared to the progress of the disease or condition without administration of the therapeutic agent.

As used herein, to “cause regression of” a disease or condition by administration of a therapeutic agent means that the progress of at least one detectable physical characteristic or symptom of the disease or condition is reversed to some extent following administration of the therapeutic agent.

A subject, including but not limited to a human subject, having a predisposition for or in need of prevention may be identified by the skilled practitioner by established methods and criteria in the field given the teachings herein. The skilled practitioner may also readily diagnose individuals as in need of inhibition or treatment based upon established criteria in the field for identifying angiogenesis and/or neovascularization given the teachings herein.

As used herein, a “subject” is generally any animal that may benefit from administration of the therapeutic agents described herein. In some variations the therapeutic agents are administered to a mammalian subject. In some variations the therapeutic agents are administered to a human subject. In some variations the therapeutic agents may be administered to a veterinary animal subject. In some variations the therapeutic agents may be administered to a model experimental animal subject.

An “effective amount,” which is also referred to herein as a “therapeutically effective amount,” of a therapeutic agent for administration as described herein is that amount of the therapeutic agent that provides the therapeutic effect sought when administered to the subject, including but not limited to a human subject. The achieving of different therapeutic effects may require different effective amounts of therapeutic agent. For example, the therapeutically effective amount of a therapeutic agent used for preventing a disease or condition may be different from the therapeutically effective amount used for treating, inhibiting, delaying the onset of, or causing the regression of the disease or condition. In addition, the therapeutically effective amount may depend on the age, weight, and other health conditions of the subject as is well know to those versed in the disease or condition being addressed. Thus, the therapeutically effective amount may not be the same in every subject to which the therapeutic agent is administered.

An effective amount of a therapeutic agent for treating, preventing, inhibiting, delaying the onset of, or causing the regression of a specific disease or condition is also referred to herein as the amount of therapeutic agent effective to treat, prevent, inhibit, delay the onset of, or cause the regression of the disease or condition.

Nonlimiting examples of ways to determine whether a level of therapeutic agent is a “therapeutically effective amount” to treat, prevent, inhibit, delay on set of, or cause the regression of the diseases and conditions described in the Diseases and Conditions section, a solid drug delivery system may be administered in in vitro or animal models for the diseases or conditions of interest, and the effects may be observed. In addition, dose ranging human clinical trials may be conducted to determine the therapeutically effective amount of a therapeutic agent.

Routes of Administration

The solid drug delivery systems described herein may be administered to a subject, including but not limited to a human subject, by one or more of the routes of administration described herein.

The solid drug delivery systems described herein may be placed in a subject or to the eye of a subject, including placement subtenon, posterior juxtascleral, or in or proximal to the conjunctiva, in or proximal to the area between the sclera and conjunctiva, or in or proximal to the sclera of the human subject. The solid drug delivery system so placed may deliver the therapeutic agent

In some variations, the solid drug delivery systems and methods described herein deliver one or more therapeutic agents proximal to an area where a disease or condition is to be treated, prevented, inhibited, onset delayed, or regression caused.

In some variations, the solid drug delivery systems and methods described herein deliver one or more therapeutic agents to an eye of a subject, including the macula and the retina choroid, in an amount and for a duration effective to treat, prevent, inhibit, delay the onset of, or cause the regression of the diseases and conditions described in the Diseases and Conditions section.

“Retina choroid” and “retina choroid tissues,” as used herein, are synonymous and refer to the combined retina and choroid tissues of the eye.

As a non-limiting example, the solid drug delivery systems described herein may be placed subtenon, posterior juxtascleral, or in or proximal to the conjunctiva, in or proximal to the area between the sclera and conjunctiva, or in or proximal to the sclera of the human subject, either by direct administration to these tissues or by periocular routes, in amounts and for a duration effective to treat, prevent, inhibit, delay the onset of, or cause the regression of CNV and wet AMD. The effective amounts and durations may be different for each of treating, preventing, inhibiting, delaying the onset of, or causing the regression of CNV and wet AMD, and for each of the different sites of delivery. For a description of exemplary periocular routes for retinal drug delivery, see Periocular routes for retinal drug delivery, Raghava et al. (2004), Expert Opin. Drug Deliv. 1(1):99-114, which is incorporated herein by reference in its entirety.

Routes of administration include but are not limited to placement of the solid drug delivery system via forceps or by injection into a medium in the body, including but not limited to intraocular and periocular placement.

Intravitreal administration is more invasive than some other types of ocular procedures. Because of the potential risks of adverse effects, intravitreal administration may not be optimal for treatment of relatively healthy eyes. By contrast, periocular administration, such as subconjunctival administration, is much less invasive than intravitreal administration. When a therapeutic agent is delivered by a periocular route, it may be possible to treat patients with healthier eyes than could be treated using intravitreal administration. In some variations, subconjunctival placement is used to prevent or delay onset of a disease or condition of the eye, where the eye of the subject has visual acuity of 20/40 or better.

“Subconjunctival” placement or injection, as used herein, refers to placement or injection between the sclera and conjunctiva. Subconjunctival is sometimes referred to herein as “sub-conj” administration. Subconjunctival delivery may be by placement or injection of a solid drug delivery system comprising a therapeutic agent underneath the conjunctiva, or in the area between the sclera and conjunctiva. Local pressure to the subconjunctival site of therapeutic agent placement may elevate delivery of the therapeutic agent to the posterior segment by reducing local choroidal blood flow.

In some variations the solid drug delivery systems described herein may be placed by injection or placement using forceps. In some variations the solid drug delivery systems may be placed in various positions within the ocular, periocular or other region for delivery to a subject or to the eye of a subject. In some variations, solid drug delivery systems up to 2 mm thick and 5 mm long are placed in or proximal to the eye of a subject.

Placement of the solid drug delivery system comprising a therapeutic agent into the vitreous may provide a high local concentration of therapeutic agent in the vitreous and retina. Further, it has been found that in the vitreous the clearance half-lives of drugs increases with molecular weight.

Intracameral delivery, e.g. by placement or injection into the anterior chamber of they eye, may also be used.

Subtenon placement may be by placement or injection of therapeutic agent into the tenon's capsule around the upper portion of the eye and into the “belly” of the superior rectus muscle.

Retrobulbar placement refers to placement, e.g. by injection, into the conical compartment of the four rectus muscles and their intermuscular septa, behind the globe of the eye.

Peribulbar placement may be at a location external to the confines of the four rectus muscles and their intramuscular septa, i.e., outside of the muscle cone.

Posterior juxtascleral placement refers to placement of a therapeutic agent near and above the macula, in direct contact with the outer surface of the sclera, and without puncturing the eyeball.

For a description of exemplary methods or sites for placement or injection via periocular routes for retinal drug delivery, see Periocular routes for retinal drug delivery, Raghava et al. (2004), Expert Opin. Drug Deliv. 1(1):99-114, which is incorporated herein by reference in its entirety.

In some variations the solid drug delivery system is placed in an ocular region for transscleral delivery by a variety of means including but not limited to placement inside a surgically formed scleral flap, and placement proximal to the outer scleral surface. Positions in which the solid polymer implant may be placed include but are not limited to subconjunctival placement, subtenon placement, and intrascleral placement.

In some variations of placement in a scleral flap, either in the clinic, procedure room, or operating room the eye may be prepared in a standard preoperative manner, the sclera will be exposed, and the creation of the flap is performed with an appropriate blade. A suture may or may not be required. In some variations of placement proximal to the outer scleral surface, either in the clinic, procedure room, or operating room the eye is prepared in a standard preoperative manner, the sclera is exposed, and the solid polymer implant placed on or attached to the outer surface of the sclera.

Routes of administration that may be used to administer a solid drug delivery system include but are not limited to placement of the solid drug delivery systems into the eye of a subject, including but not limited to a human subject. The solid drug delivery systems may be administered systemically, including but not limited to the following delivery routes: rectal, vaginal, infusion, intramuscular, intraperitoneal, intraarterial, intrathecal, intrabronchial, intracisternal, cutaneous, subcutaneous, intradermal, transdermal, intravenous, intracervical, intraabdominal, intracranial, intraocular, intrapulmonary, intrathoracic, intratracheal, nasal, buccal, sublingual, oral, parenteral, or nebulised or aerosolized using aerosol propellants.

Solid drug delivery systems comprising one or more therapeutic agents can be administered directly to the eye using a variety of procedures, including but not limited to procedures in which (1) the solid drug delivery system is administered by injection using a syringe and hypodermic needle, (2) a specially designed device is used to place the solid drug delivery system, (3) prior to placement of the solid drug delivery system, a pocket is surgically formed within the sclera to serve as a receptacle for the solid drug delivery system. For example, in one administration procedure a surgeon forms a pocket within the sclera of the eye followed by placement of a solid drug delivery system comprising the therapeutic agent into the pocket.

Other administration procedures include, but are not limited to procedures in which a solid drug delivery system comprising a therapeutic agent is placed near one or more of the retina choroid or the macula.

When the therapeutic agent is rapamycin, the solid drug delivery systems may be used to deliver or maintain an effective amount of rapamycin in the vitreous. In one nonlimiting example, it is believed that a delivery system delivering rapamycin in an amount capable of providing a concentration of rapamycin of 10 pg/ml to 2 μg/ml in the vitreous may be used for treatment of wet AMD. In another nonlimiting example, it is believed that a delivery system delivering a concentration of rapamycin of 0.01 pg/mg to 10 ng/mg in the retina choroid may be used for treatment of wet AMD. Other effective concentrations are readily ascertainable by those of skill in the art.

One method that may be used to deliver the solid drug delivery systems described herein is delivery by injection. In this method solid drug delivery systems may be injected or implanted into a subject, including but not limited to a human subject, or into a position in or proximal to an eye of the subject for delivery to a subject or to the eye of a subject. Injection includes but is not limited to intraocular and periocular placement or injection.

A “periocular” route of administration means placement near or around the eye. Nonlimiting examples of placement positions that are in or proximal to an eye of a subject include intracameral, periocular, limited to subconjunctival, subtenon, retrobulbar, peribulbar and posterior juxtascleral delivery.

Subconjunctival placement may be by injection or other placement of the solid drug delivery system comprising the therapeutic agent underneath the conjunctiva, or between the sclera and conjunctiva. Subtenon placement may be by injection or other placement of the solid drug delivery system comprising the therapeutic agent into the tenon's capsule around the upper portion of the eye and into the “belly” of the superior rectus muscle. Retrobulbar placement may be by injection or other placement of the solid drug delivery system into the conical compartment of the four rectus muscles and their intermuscular septa, behind the globe of the eye. Peribulbar placement may be at a location external to the confines of the four rectus muscles and their intramuscular septa, i.e., outside of the muscle cone. Posterior juxtascleral delivery may be by placement of a therapeutic agent near and above the macula, in direct contact with the outer surface of the sclera, and without puncturing the eyeball.

In some variations the solid drug delivery systems described herein are placed intraocularly. Intraocular placement includes placement within the eye.

Sites to which the solid drug delivery systems may be administered include but are not limited to the vitreous, aqueous humor, sclera, conjunctiva, between the sclera and conjunctiva, the retina choroid, the outer surface of the sclera, the macula, or other area in or proximal to the eye of a subject. Methods that may be used for placement of the solid drug delivery systems include but are not limited to injection.

When the therapeutic agent is rapamycin, the solid drug delivery systems may be used to deliver or maintain an amount of rapamycin in tissues of the eye, including without limitation retina, choroid, or the vitreous, which amount is effective to treat AMD. In one nonlimiting example, it is believed that a solid drug delivery system delivering rapamycin in an amount capable of providing a concentration of rapamycin of 0.1 pg/ml to 2 μg/ml in the vitreous may be used for treatment of wet AMD. In some nonlimiting examples, it is believed that a solid drug delivery systems delivering a concentration of rapamycin of 0.1 pg/mg to 1 μg/mg in the retina choroid may be used for treatment of wet AMD. Other effective concentrations are readily ascertainable by those of skill in the art based on the teachings described herein.

Intravitreal and Subconjunctival Solid Drug Delivery System Placement for Delivery of Rapamycin for Treatment of AMD

In one method described herein, a solid drug delivery system comprising rapamycin is placed subconjunctivally to prevent, treat, inhibit, delay onset of, or cause regression of angiogenesis in the eye, such as to prevent, treat, inhibit, delay onset of, or cause regression of CNV as observed, for example, in AMD. Rapamycin has been shown to inhibit CNV in rat and mice models, as described in U.S. application Ser. No. 10/665,203, which is incorporated herein by reference in its entirety. Rapamycin has been observed to inhibit Matrigel™ and laser-induced CNV when administered systemically and subretinally. Also, periocular injection of rapamycin inhibits laser-induced CNV.

Other therapeutic agents that may be delivered to the eye, particularly the vitreous of an eye, for treatment, prevention, inhibition, delaying onset, or causing regression of angiogenesis in the eye (such as CNV) are members of the limus family of compounds other than rapamycin including but not limited to everolimus and tacrolimus (FK-506).

As described herein, the dosage of the therapeutic agent will depend on the condition being addressed, whether the condition is to be treated, prevented, inhibited, have onset delayed, or be caused to regress, the particular therapeutic agent, and other clinical factors such as weight and condition of the subject and the route of administration of the therapeutic agent. It is to be understood that the methods and solid drug delivery systems described herein have application for both human and veterinary use, as well as uses in other possible animals. In some variations the concentration of rapamycin used in the methods described herein is one that provides 0.1 pg/mg or pg/mg or more of rapamycin at the tissue level; 1 pg/ml or ng/mg or more at the tissue level; 0.01 ng/ml or ng/mg or more at the tissue level; 0.1 ng/ml or ng/mg or more at the tissue level; 0.5 ng/ml or ng/mg or more at the tissue level; 1 ng/ml or more at the tissue level; 2 ng/ml or more at the tissue level, 3 ng/ml or more at the tissue level; 5 ng/ml or more at the tissue level; 10 ng/ml or more at the tissue level; 15 ng/ml or more at the tissue level; 20 ng/ml or more at the tissue level; 30 ng/ml or more at the tissue level; or 50 ng/ml or more at the tissue level. One of ordinary skill in the art would know how to arrive at an appropriate concentration depending on the route and duration of administration utilized.

Generally, the amount of rapamycin administered in a solid drug delivery system is an amount sufficient to treat, prevent, inhibit, delaying the onset, or cause regression of the disease or condition of the eye for the required amount of time.

In one method, a solid drug delivery system as described herein containing an amount of rapamycin of between 20 μg and 10 mg is administered to a human subject for treatment of wet AMD. In another method, an amount of rapamycin of between 30 μg and 9 mg is administered to a human subject for treatment of wet AMD. In another method, an amount of rapamycin of between 10 μg and 90 μg is administered to a human subject for treatment of wet AMD. In another method, an amount of rapamycin of between 60 μg and 120 μg is administered to a human subject for treatment of wet AMD. In another method, an amount of rapamycin of between 100 μg and 400 μg is administered to a human subject for treatment of wet AMD. In another method, an amount of rapamycin of between 400 μg and 1 mg is administered to a human subject for treatment of wet AMD. In another method, an amount of rapamycin of between 1 mg and 5 mg is administered to a human subject for treatment of wet AMD. In another method, an amount of rapamycin of between 3 mg and 7 mg is administered to a human subject for treatment of wet AMD. In another method, an amount of rapamycin of between 5 mg and 10 mg is administered to a human subject for treatment of wet AMD.

In another method, a solid drug delivery system as described herein containing an amount of rapamycin of between 20 μg and 10 mg is administered to a human subject for treatment of angiogenesis, including but not limited to choroidal neovascularization. In another method, an amount of rapamycin of between 30 μg and 9 mg is administered to the human subject; in another method, an amount of rapamycin of between 10 μg and 90 μg is administered to the human subject; in another method, an amount of rapamycin of between 60 μg and 120 μg is administered to the human subject; in another method, an amount of rapamycin of between 100 μg and 400 μg is administered to the human subject; in another method, an amount of rapamycin of between 400 μg and 1 mg is administered to the human subject; in another method, an amount of rapamycin of between 1 mg and 5 mg is administered to the human subject; in another method, an amount of rapamycin of between 3 mg and 7 mg is administered to the human subject; in another method, an amount of rapamycin of between 5 mg and 10 mg is administered to the human subject.

In one method, a solid drug delivery system as described herein containing an amount of a therapeutic agent equivalent to an amount of rapamycin of between 20 μg and 10 mg is administered to a human subject for treatment of wet AMD. In another method, an amount of a therapeutic agent equivalent to an amount of rapamycin of between 30 μg and 9 mg is administered to the human subject; in another method, an amount of a therapeutic agent equivalent to an amount of rapamycin of between 10 μg and 90 μg is administered to the human subject; in another method, an amount of a therapeutic agent equivalent to an amount of rapamycin of between 60 μg and 120 μg is administered to the human subject; in another method, an amount of a therapeutic agent equivalent to an amount of rapamycin of between 100 μg and 400 μg is administered to a human subject for treatment of wet AMD. In another method, an amount of a therapeutic agent equivalent to an amount of rapamycin of between 400 μg and 1 mg is administered to the human subject; in another method, an amount of a therapeutic agent equivalent to an amount of rapamycin of between 1 mg and 5 mg is administered to the human subject; in another method, an amount of a therapeutic agent equivalent to an amount of rapamycin of between 3 mg and 7 mg is administered to the human subject; in another method, an amount of a therapeutic agent equivalent to an amount of rapamycin of between 5 mg and 10 mg is administered to the human subject.

In another method, a solid drug delivery system as described herein containing an amount of a therapeutic agent equivalent to an amount of rapamycin of between 20 μg and 10 mg is administered to a human subject for treatment of angiogenesis, including but not limited to choroidal neovascularization. In another method, an amount of a therapeutic agent equivalent to an amount of rapamycin of between 30 μg and 9 mg is administered to the human subject; in another method, an amount of a therapeutic agent equivalent to an amount of rapamycin of between 10 μg and 90 μg is administered to the human subject; in another method, an amount of a therapeutic agent equivalent to an amount of rapamycin of between 60 μg and 120 μg is administered to the human subject; in another method, an amount of a therapeutic agent equivalent to an amount of rapamycin of between 100 μg and 400 μg is administered to the human subject; in another method, an amount of a therapeutic agent equivalent to an amount of rapamycin of between 400 μg and 1 mg is administered to the human subject; in another method, an amount of a therapeutic agent equivalent to an amount of rapamycin of between 1 mg and 5 mg is administered to the human subject; in another method, an amount of a therapeutic agent equivalent to an amount of rapamycin of between 3 mg and 7 mg is administered to the human subject; in another method, an amount of a therapeutic agent equivalent to an amount of rapamycin of between 5 mg and 10 mg is administered to the human subject.

Delivery of the therapeutic agents described herein may, for example, be delivered at a dosage range between 1 ng/day and 100 μg/day, or at dosages higher or lower than this range, depending on the route and duration of administration. In some variations of solid drug delivery system used in the methods described herein, the therapeutic agents are delivered at a dosage range of between 0.1 μg/day and 10 μg/day. In some variations of solid drug delivery system used in the methods described herein, the therapeutic agents are delivered at a dosage range of between 1 μg/day and 5 μg/day. Dosages of various therapeutic agents for treatment, prevention, inhibition, delay of onset, or cause of regression of various diseases and conditions described herein can be refined by the use of clinical trials. Additionally, dose ranges include those disclosed in U.S. Pat. Nos. 6,376,517 and 5,387,589, the contents of which are hereby incorporated by reference in their entirety.

The solid drug delivery systems described herein may be used for placement in the eye, particularly proximal to the conjunctiva, between the sclera and conjunctiva, in a surgically introduced scleral flap, attached or adhered to the sclera, or otherwise proximal to the sclera; or placement subtenon, posterior juxtascleral, retrobulbar or proximal to the superior rectus muscle, of therapeutically effective amounts of rapamycin for extended periods of time to treat, prevent, inhibit, delay the onset of, or cause regression of CNV, and thus may be used to treat, prevent, inhibit, delay the onset of, or cause regression of wet AMD. It is believed that by changing certain characteristics of the solid drug delivery systems described herein, including but not limited to the shape, size, positioning and components of the solid drug delivery systems, the solid drug delivery systems described herein may be used to deliver therapeutically effective amounts of rapamycin to the eye for a variety of extended time periods including delivery of therapeutic amounts for at least 30 days, at least 60 days, at least 90 days, at least 120 days, at least 150 days, at least 180 days, at least 210 days, at least 240 days, at least 270 days, at least 300 days, at least 330 days, or at least 360 days.

For treatment, prevention, inhibition, delaying the onset of, or causing the regression of certain diseases or conditions, it may be desirable to maintain delivery of a therapeutically effective amount of the therapeutic agent for an extended period of time. Depending on the disease or condition being treated, prevented, inhibited, having onset delayed, or being caused to regress this extended period of time may be at least 1 week, at least 2 weeks, at least 3 weeks, at least 1 month, at least 3 months, at least 6 months, at least 9 months, or at least 1 year. Generally, however, any extended period of delivery may be possible. A therapeutically effective amount of agent may be delivered for an extended period by a solid drug delivery system that maintains for the extended period a concentration of agent in a subject or an eye of a subject sufficient to deliver a therapeutically effective amount of agent for the extended time.

Delivery of a therapeutically effective amount of the therapeutic agent for an extended period may be achieved via placement of one solid drug delivery system or may be achieved by application of two or more doses of solid drug delivery systems. As a non-limiting example of such multiple applications, maintenance of the therapeutic amount of rapamycin for 3 months for treatment, prevention, inhibition, delay of onset, or cause of regression of wet AMD may be achieved by placement of one solid drug delivery system delivering a therapeutic amount for 3 months or by sequential application of a plurality of solid drug delivery systems. The optimal dosage regime will depend on the therapeutic amount of the therapeutic agent needing to be delivered, and the period over which it need be delivered. Those versed in such extended therapeutic agent delivery dosing will understand how to identify dosing regimes that may be used based on the teachings provided herein.

Delivery of the therapeutic agents described herein may, for example, be delivered at a dosage range between 1 ng/day and 100 μg/day, or at dosages higher or lower than this range, depending on the route and duration of administration. In some variations of solid drug delivery systems used in the methods described herein, the therapeutic agents are delivered at a dosage range of between 0.1 μg/day and 10 μg/day. In some variations of solid drug delivery systems used in the methods described herein, the therapeutic agents are delivered at a dosage range of between 1 μg/day and 5 μg/day. Dosages of various therapeutic agents for treatment, prevention, inhibition, delay of onset, or cause of regression of various diseases and conditions described herein can be refined by the use of clinical trials.

When a therapeutically effective amount of rapamycin is administered to a subject suffering from wet AMD, the rapamycin may treat, inhibit, or cause regression of the wet AMD. Different therapeutically effective amounts may be required for treatment, inhibition or causing regression. A subject suffering from wet AMD may have CNV lesions, and it is believed that administration of a therapeutically effective amount of rapamycin may have a variety of effects, including but not limited to causing regression of the CNV lesions, stabilizing the CNV lesion, and preventing progression of an active CNV lesion.

When a therapeutically effective amount of rapamycin is administered to a subject suffering from dry AMD, it is believed that the rapamycin may prevent or slow the progression of dry AMD to wet AMD.

Methods of Administering an Anti-Proliferative Agent Proximal to an Ocular Device

In some variations, an ocular condition is treated by administering an anti-proliferative agent proximal to an ocular device.

In some variations the ocular device is a glaucoma drainage device.

Most generally, a glaucoma drainage device is any device capable of being used for draining fluid from the aqueous humor of an eye. Glaucoma Drainage Devices include but are not limited to devices that include shunts, stents, tubes, membranes and valves and combinations of these components. Glaucoma Drainage Devices include but are not limited to those described in U.S. Pat. Nos. 6,007,510 and 6,142,969, the contents of which are incorporated herein by reference in their entirety; the Optonol Ex-Press™ Miniature Glaucoma Implant (510(k) number K012852); the Ahmed Glaucoma Valve (510(k) number K980657); the OptiMed Glaucoma Shunt (510(k) number K903462); and the Baerveldt Glaucoma Shunt (510(k) numbers K905129 and K955455). Additional glaucoma drainage devices include but are not limited to those described in U.S. 60/666,872, filed Mar. 30, 2005 with attorney docket number 57796-30007.00, titled GLAUCOMA DRAINAGE DEVICES.

Once implanted in a subject, ocular devices such as glaucoma drainage devices may cause cellular proliferation that may result in the device ceasing to function, or having a reduced useful lifetime. Described herein are devices and methods for use of ocular devices with one or more anti-proliferative agents. The anti-proliferative agent may be coated onto the glaucoma drainage device, may be incorporated into materials used to make the glaucoma drainage device, or a source of anti-proliferative agent may be administered to provide the anti-proliferative agent proximal to an implanted glaucoma drainage device, as described further in U.S. 60/666,872, filed Mar. 30, 2005 with attorney docket number 57796-30007.00, titled GLAUCOMA DRAINAGE DEVICES. In the methods, devices, and compositions described herein, unless the context makes clear otherwise, one or more than one anti-proliferative agent may be used. Anti-proliferative agents that may be used are described herein, including but not limited to in the section titled Anti-Proliferative Agents for Use With Ocular Devices herein or in U.S. 60/666,872, filed Mar. 30, 2005 with attorney docket number 57796-30007.00, titled GLAUCOMA DRAINAGE DEVICES.

In some variations of the methods, devices, and compositions described herein the anti-proliferative agent is rapamycin.

Described herein are methods of using Glaucoma Drainage Devices in which the Glaucoma Drainage Device is used in combination with a source of one or more anti-proliferative agents. Generally any source may be used that is capable of delivering the anti-proliferative agent or agents in an amount, for a time period, and to a position capable of reducing cell proliferation caused by implantation of the Glaucoma Drainage Device. In addition to the solid drug delivery systems described herein, sources of anti-proliferative agent or agents that may be used include but are not limited to solid implants containing anti-proliferative agent or agents, self-emulsifying formulations, liquid formulations, solutions, suspensions, formulations capable of forming a gel containing the anti-proliferative agent or agents when the formulation is placed in a medium of the eye, in situ gelling formulations, emulsions, and formulations capable of forming a non-dispersed mass containing the anti-proliferative agent or agents when the formulation is placed in a medium of the eye. Sources of anti-proliferative agent or agents that may be used include but are not limited to the formulations and devices described in the following patent applications, each of which is incorporated herein by reference in its entirety: application Ser. No. 10/665,203, filed Sep. 18, 2003, with attorney docket number 559092000100, titled METHOD OF INHIBITING CHOROIDAL NEOVASCULARIZATION; application Ser. No. 10/945,682 filed Sep. 20, 2004, with attorney docket number 577962000100, titled TRANSSCLERAL DELIVERY; application No. 60/651,790, filed Feb. 9, 2005, with attorney docket number 577963000200, titled FORMULATIONS FOR OCULAR TREATMENT; application 60/664,040, filed Mar. 21, 2005, with attorney docket number 577963000400, titled LIQUID FORMULATIONS FOR TREATMENT OF DISEASES OR CONDITIONS; application 60/664,119, filed Mar. 21, 2005, with attorney docket number 577963000500, titled DRUG DELIVERY SYSTEMS FOR TREATMENT OF DISEASES OR CONDITIONS; application 60/664,306, filed Mar. 21, 2005, with attorney docket number 577963000600, titled 1N SITU GELLING FORMULATIONS AND LIQUID FORMULATIONS FOR TREATMENT OF DISEASES OR CONDITIONS; application Ser. No. 11/351,844 filed Feb. 9, 2006, with attorney docket number 57796-2000200, titled FORMULATIONS FOR OCULAR TREATMENT; application Ser. No. 11/351,761 filed Feb. 9, 2006, with attorney docket number 57796-2000400, titled LIQUID FORMULATIONS FOR TREATMENT OF DISEASES OR CONDITIONS; and application 60/772,018 filed Feb. 9, 2006, with attorney docket number 57796-3001000, titled STABLE FORMULATIONS.

The source of anti-proliferative agent may be separate from or attached to the Glaucoma Drainage Device. As a nonlimiting example, the source of anti-proliferative agent may be any solid drug delivery device or other formulation capable of releasing the anti-proliferative agent and the solid structure may be attached to or incorporated into the Glaucoma Drainage Device. Solid structures capable of releasing the anti-proliferative agent that may be used include but are not limited to an anti-proliferative agent containing reservoir.

In some variations, the solid drug delivery systems described herein are used to treat glaucoma. In some variations, the solid drug delivery systems described herein for treating glaucoma comprise a limus compound such as rapamycin, and are used as a surgical adjuvant to prevent, reduce or delay surgical complications. In some variations, the solid drug delivery systems described herein for treating glaucoma comprise a limus compound such as rapamycin, and are used to improve or prolong surgical implant success. In some variations, the solid drug delivery systems described herein for treating glaucoma comprise a limus compound such as rapamycin, and are used to improve or prolong success of an argon laser trabeculectomy or other glaucoma-related surgery. In some variations, the solid drug delivery systems described herein have a neuroprotective effect and are used to treat glaucoma.

The source of anti-proliferative agent may be placed in the appropriate position in the eye using any method capable of placing the source including but not limited to by injection and by placement of a solid drug delivery device. In one device-source combination described herein, the source of anti-proliferative agent may be placed outside of the sclera and may deliver the anti-proliferative agent transsclerally.

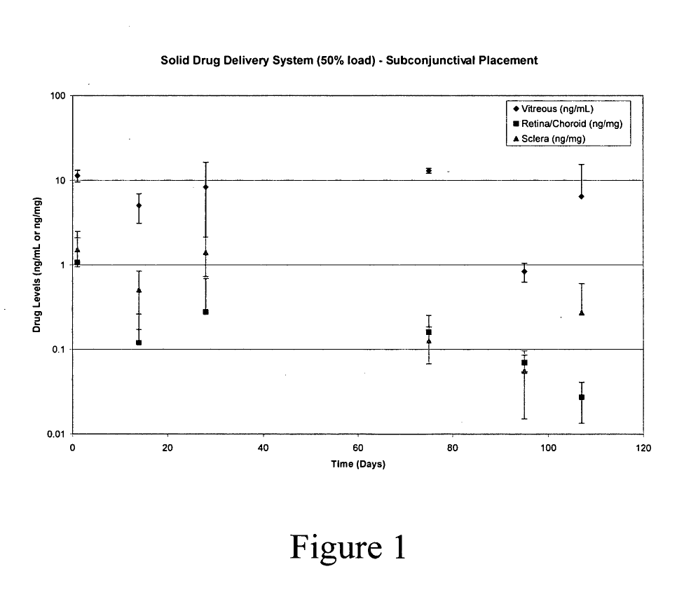

In one combination device and anti-proliferative agent source described herein, the device has the form of a device as shown in FIG. 1 of U.S. Pat. No. 6,142,969, or other similar version of the device as shown in other Figures in this patent. In one such device source combination, the source of anti-proliferative agent is a solid device that may be placed proximal to the tube end labeled 16. In another such device-source combination, the source of anti-proliferative agent is a solid device that may be placed proximal to the portion labeled 24 in FIG. 1. In another such device source combination, the source of anti-proliferative agent is a structure that is permeable or semi-permeable to the fluids of the aqueous humor and that is placed inside the tube labeled 12 in FIG. 1. In one such device, the permeable or semi-permeable structure is placed proximal to the tube end labeled 16 in FIG. 1. In one such device, the permeable or semi-permeable structure is a mesh-like structure or sponge like structure, or a porous foam structure that incorporates the anti-proliferative agent and thus can act as a source of the anti-proliferative agent.

Also described herein are kits containing any of the Glaucoma Drainage Devices described herein and any one or more of the anti-proliferative agent sources described herein.

Also described herein are methods of draining fluid from the aqueous humor by use of a Glaucoma Drainage Device described herein together with use of any one or more of the anti-proliferative agent sources described herein. Generally the combinations of the Glaucoma Drainage Devices and sources of anti-proliferative agent sources described herein can be used for addressing any of the diseases or conditions that may be addressed using the Glaucoma Drainage Devices alone, including but not limited to those diseases and conditions described in the articles in U.S. 60/666,872 and U.S. Pat. Nos. 6,007,510 and 6,142,969.

In some variations, the formulations or solid drug delivery devices comprising an anti-proliferative agent deliver an amount of the therapeutica gent effective to reduce cellular proliferation proximal to the ocular device for a period of at least 14, at least 30, at least 45, at least 60, at least 90, or at least 105 days following placement of the solid drug delivery system proximal to the ocular device.

Anti-Proliferative Agents for Use with Ocular Devices

In some variations, the methods and formulations described herein comprise an anti-proliferative agent. In some variations the anti-proliferative agent is any anti-proliferative agent which has the desired effect. In some variations the anti-proliferative agent is any anti-proliferative agent described in the Therapeutic Agents section. In some variations the anti-proliferative agent is a limus compound or an immunophilin binding compound as described in the Therapeutic Agents section. In some variations the anti-proliferative agent is a steroidal agent as described in the Therapeutic Agents section, and in some variations the steroidal agent is present in the amounts described in the Therapeutic Agents section. In some variations the anti-proliferative agent is a combination of therapeutic agents. In some variations the anti-proliferative agent is used in combination with one or more other therapies or therapeutic agents, including but not limited to those therapeutic agents listed for combination therapy in the Therapeutic Agents section.

In some variations the antiproliferative agent is one or more of those disclosed in the following patents and publications, the contents of each of which is incorporated herein by reference in its entirety: PCT publication WO 2004/027027, published Apr. 1, 2004, titled Method of inhibiting choroidal neovascularization, assigned to Trustees of the University of Pennsylvania; U.S. Pat. No. 5,387,589, issued Feb. 7, 1995, titled Method of Treating Ocular Inflammation, with inventor Prassad Kulkarni, assigned to University of Louisville Research Foundation; U.S. Pat. No. 6,376,517, issued Apr. 23, 2003, titled Pipecolic acid derivatives for vision and memory disorders, assigned to GPI NIL Holdings, Inc; PCT publication WO 2004/028477, published Apr. 8, 2004, titled Method subretinal administration of therapeutics including steroids: method for localizing pharmadynamic action at the choroid and retina; and related methods for treatment and or prevention of retinal diseases, assigned to Innorx, Inc; U.S. Pat. No. 6,416,777, issued Jul. 9, 2002, titled Ophthalmic drug delivery device, assigned to Alcon Universal Ltd; U.S. Pat. No. 6,713,081, issued Mar. 30, 2004, titled Ocular therapeutic agent delivery device and methods for making and using such devices, assigned to Department of Health and Human Services; U.S. Pat. No. 5,100,899, issued Mar. 31, 1992, titled Methods of inhibiting transplant rejection in mammals using rapamycin and derivatives and prodrugs thereof.

In some variations the antiproliferative agent is one or more of pyrrolidine, dithiocarbamate (NFκB inhibitor); squalamine; TPN 470 analogue and fumagillin; PKC (protein kinase C) inhibitors; Tie-1 and Tie-2 kinase inhibitors; inhibitors of VEGF receptor kinase; proteosome inhibitors such as Velcade™ (bortezomib, for injection; ranibuzumab (Lucentis™) and other antibodies directed to the same target; pegaptanib (Macugen™); vitronectin receptor antagonists, such as cyclic peptide antagonists of vitronectin receptor-type integrins; α-v/β-3 integrin antagonists; α-v/β-1 integrin antagonists; thiazolidinediones such as rosiglitazone or troglitazone; interferon, including γ-interferon or interferon targeted to CNV by use of dextran and metal coordination; pigment epithelium derived factor (PEDF); endostatin; angiostatin; tumistatin; canstatin; anecortave acetate; acetonide; triamcinolone; tetrathiomolybdate; RNA silencing or RNA interference (RNAi) of angiogenic factors, including ribozymes that target VEGF expression; Accutane™ (13-cis retinoic acid); ACE inhibitors, including but not limited to quinopril, captopril, and perindozril; inhibitors of mTOR (mammalian target of rapamycin); 3-aminothalidomide; pentoxifylline; 2-methoxyestradiol; colchicines; AMG-1470; cyclooxygenase inhibitors such as nepafenac, rofecoxib, diclofenac, rofecoxib, NS398, celecoxib, vioxx, and (E)-2-alkyl-2(4-methanesulfonylphenyl)-1-phenylethene; t-RNA synthase modulator; metalloprotease 13 inhibitor; acetylcholinesterase inhibitor; potassium channel blockers; endorepellin; purine analog of 6-thioguanine; cyclic peroxide ANO-2; (recombinant) arginine deiminase; epigallocatechin-3-gallate; cerivastatin; analogues of suramin; VEGF trap molecules; apoptosis inhibiting agents; Visudyne™, snET2 and other photo sensitizers, which may be used with photodynamic therapy (PDT); inhibitors of hepatocyte growth factor (antibodies to the growth factor or its receptors, small molecular inhibitors of the c-met tyrosine kinase, truncated versions of HGF e.g. NK4).

### EXAMPLES

### Example 1

### Rapamycin-Containing Solid Drug Delivery System

A solid drug delivery system comprising rapamycin was prepared with the following components, where the percent is the weight of the component per weight of the total: 47.7% rapamycin (obtained from LC laboratories (Woburn, Mass.), and Chunghwa Chemical Synthesis & BioTech. Co, Ltd (Taiwan)), 23.25% PVP K90 (obtained from BASF), 23.25% Eudragit RL100 (obtained from Rohm Pharma Polymers), and 5.8% PEG 400 (obtained from DOW Chemical). Briefly, Eudragit RL 100 was added to a mixture of pure ethanol and PEG 400 in a bottle. Eudragit RL 100 was dissolved by vigorous shaking using a vortex mixer. PVP 90 was added to the solution of PEG 400, Ethanol and Eudragit RL 100. PVP 90 was also dissolved using vortex mixer. Rapamycin was added finally and mixed well to get a uniform solution. This solution was viscous and sticky. The solution was cast as a film on a silicone-coated polyester film, using a roll-over Gardner Knife at desired thickness. The wet film was dried under the hood overnight to drive off (evaporate) ethanol. Once the ethanol was driven off, the film was dried and peeled off. The film was die-cut into wafers at desired diameter circles.

### Example 2

### Subconjunctival Placement of a Rapamycin-Containing Solid Drug Delivery System

Approximately 1.5-2.5 mg of the solid drug delivery system described in Example 1 were placed into the area between the sclera and the conjunctiva of the eye of New Zealand white rabbits. Briefly, the solid drug delivery system was inserted into the subconjunctival space by a small cut of the conjunctiva with a vannas scissor, and inserted using tying forceps. After the solid drug delivery system was placed under the conjunctiva, the conjunctiva was closed with one or two sutures.

FIG. 1 depicts the level of rapamycin present in the vitreous (ng/ml), retina choroid (ng/mg), and sclera (ng/mg) at 1, 14, 28, 75, 95, and 107 days after placement of the solid drug delivery system.

The analysis was performed by LCMS (liquid chromatography mass spectroscopy) after dissection of the eye into the tissues specified below. Day 1, 14 and 28 timepoints represents the average of two eyes of each of two rabbits (four eyes at each timepoint); the day 75 timepoint represents the average of two eyes of one rabbit; the day 95 timepoint represents the average of two eyes of one rabbit and one eye of another rabbit; the day 107 timepoint represents the average of two eyes of one rabbit.

The full vitreous was homogenized and analyzed. The average concentration of the vitreous was calculated by dividing the mass of rapamycin measured by the volume of vitreous analyzed. The sample did not include the solid drug delivery system; thus, this measurement indicated the level of rapamycin delivered to the vitreous via the solid drug delivery system.

The average level of rapamycin in the vitreous at 1, 14, 28, 75, 95 and 107 days after subconjunctival placement was about 11.35, about 5.025, about 8.325, about 13, 0.83, and about 6.43 ng/ml, respectively.

The full retina choroid was homogenized and analyzed. The average concentration of the retina choroid was calculated by dividing the mass of rapamycin measured by the mass of retina choroid analyzed. The sample did not include the solid drug delivery system; thus, this measurement indicated the level of rapamycin delivered to the retina choroid via the solid drug delivery system.

The average level of rapamycin in the retina choroid at 1, 14, 28, 75, 95, and 107 days after subconjunctival placement of the solid drug delivery system was about 1.0716, 0.11975, about 0.27775, about 0.161, about 0.07, and about 0.037 ng/mg, respectively.

The sclera was analyzed in the same way as the retina choroid. The scleral sample may have included the solid drug delivery system; thus, this measurement likely indicated clearance of rapamycin from the sclera, but some inaccuracy may have been introduced due to sampling.

The average level of rapamycin in the sclera at 1, 14, 28, 75, 95 and 107 days after subconjunctival placement of the solid drug delivery system was about 1.517, 0.51, 1.40675, 0.1265, 0.06, and 0.27 ng/mg, respectively.

### Example 3

### Rapamycin-Containing Solid Drug Delivery System

A solid drug delivery system comprising rapamycin was prepared with the following components, where the percent is per weight of the total: 10.2% rapamycin, 89.8% PVP K90. Rapamycin and PVP K-90 were dissolved in ethanol, a film was cast on a release coated paper, dried to evaporate the solvent, and the resultant wafer was die-cut into its size and shape.

### Example 4

### Subconjunctival Placement of a Rapamycin-Containing Solid Drug Delivery System

Approximately 1 mg of the solid drug delivery system described in Example 3 were placed into the area between the sclera and the conjunctiva of the eye of New Zealand white rabbits as described in Example 2.

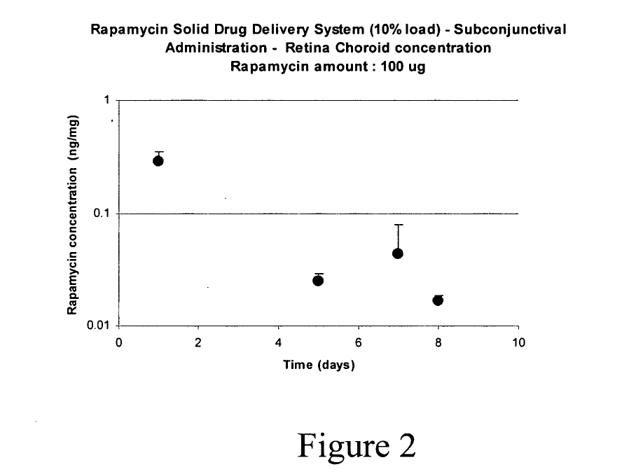

FIG. 2 depicts the level of rapamycin present in the vitreous (ng/ml) at 1, 5, 7 and 8 days after placement of the solid drug delivery system.

The analysis was by liquid chromatography and mass spectroscopy. All timepoints represents the average of two eyes of one rabbit.

The full vitreous was homogenized and analyzed. The average concentration of the vitreous was calculated by dividing the mass of rapamycin measured by the volume of vitreous analyzed. The sample did not include the solid drug delivery system; thus, this measurement indicated the level of rapamycin delivered to the vitreous via the solid drug delivery system.

The average level of rapamycin in the vitreous at 1, 5, 7 and 8 days after subconjunctival placement of the solid drug delivery system was about 68.70, 5.50, 0.80, and 0.85 ng/ml, respectively.

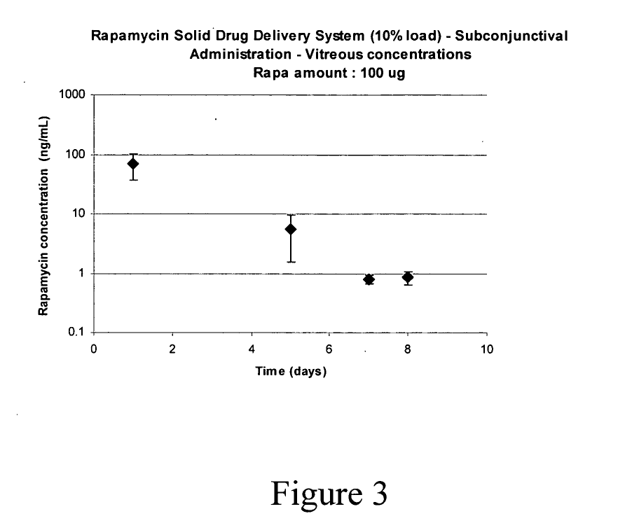

FIG. 3 depicts the level of rapamycin present in the retina choroid (ng/mg) at 1, 5, 7, and 8 days after placement of the solid drug delivery system.

The full retina choroid was homogenized and analyzed. The average concentration of the retina choroid was calculated by dividing the mass of rapamycin measured by the mass of retina choroid analyzed. The sample did not include the solid drug delivery system; thus, this measurement indicated the level of rapamycin delivered to the retina choroid via the solid drug delivery system.

The average level of rapamycin in the retina choroid at 1, 5, 7, and 8 days after subconjunctival placement of the solid drug delivery system was about 0.285, 0.025, 0.0435, and 0.0165 ng/mg, respectively.

### Example 5

### Rapamycin-Containing Solid Drug Delivery System

A solid drug delivery system comprising rapamycin was prepared with the following components, where the percent is the weight of the component per weight of the total: 45.13% rapamycin (obtained from LC laboratories (Woburn, Mass.), and Chunghwa Chemical Synthesis & BioTech. Co, Ltd (Taiwan)), 40.03% PVP K90 (obtained from BASF), 9.7% Eudragit RL100 (obtained from Rohm Pharma Polymers), and 5.14% PEG 400 (obtained from DOW Chemical). This solid drug delivery system was prepared as in Example 1.

### Example 6

### Subconjunctival Placement of a Rapamycin-Containing Solid Drug Delivery System

Approximately 1.5-2.5 mg of the solid drug delivery system described in Example 5 were placed between the sclera and the conjunctiva of the eye of New Zealand white rabbits as described in Example 2.

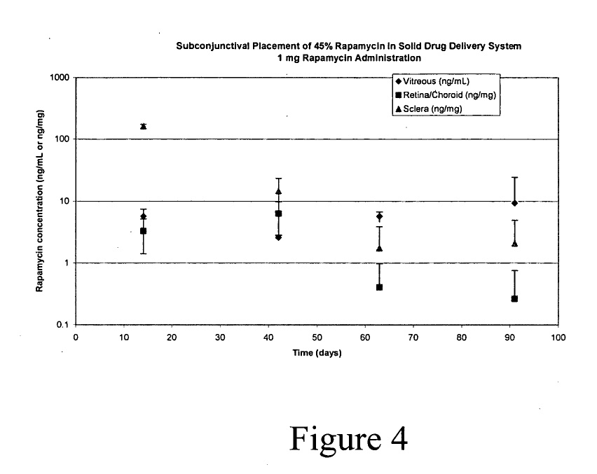

FIG. 4 depicts the level of rapamycin present in the vitreous (ng/ml), retina choroid (ng/mg), and sclera (ng/mg) at 14, 42, 63, and 91 days after placement of the solid drug delivery system.

The analysis was performed by LCMS (liquid chromatography-mass spectroscopy). Day 14, 42, 63, and 91 timepoints each represent the average of two eyes of each of two rabbits (four eyes at each timepoint).

The full vitreous was homogenized and analyzed. The average concentration of the vitreous was calculated by dividing the mass of rapamycin measured by the volume of vitreous analyzed. The sample did not include the solid drug delivery system; thus, this measurement indicated the level of rapamycin delivered to the vitreous via the solid drug delivery system.

The average level of rapamycin in the vitreous at 14, 42, 63, and 91 days after subconjunctival placement of the solid drug delivery systemation was about 5.7, about 2.6, about 5.7, and about 9.3 ng/ml, respectively.

The full retina choroid was homogenized and analyzed. The average concentration of the retina choroid was calculated by dividing the mass of rapamycin measured by the mass of retina choroid analyzed. The sample did not include the solid drug delivery system; thus, this measurement indicated the level of rapamycin delivered to the retina choroid via the solid drug delivery system.

The average level of rapamycin in the retina choroid at 14, 42, 63, and 91 days after subconjunctival placement of the solid drug delivery system was about 3.3, 6.3, about 0.41, and about 0.27 ng/mg, respectively.

The sclera was analyzed in the same way as the retina choroid. The scleral sample may have included the solid drug delivery system; thus, this measurement likely indicated clearance of rapamycin from the sclera, but some inaccuracy may have been introduced due to sampling.

The average level of rapamycin in the sclera at 14, 42, 63, and 91 days after subconjunctival placement of the solid drug delivery system was about 164, 14.6, 1.75, and 2.1 ng/mg, respectively.

### Example 7

### Rapamycin-Containing Solid Drug Delivery Systems

A solid drug delivery system comprising rapamycin was prepared with the following components, where the percent is the weight of the component per the weight of the total: 19.33% rapamycin, 21.78% PVP K90, 24.56% PEG 400, and 34.33% pure ethanol. Briefly, PVP and Rapa were added to a mixture of of PEG 400 and pure ethanol in a bottle, and the mixture was vigorously mixed to obtain a uniform viscous suspension. The viscous suspension was applied onto a backing in the shape of a microcup using a spatula.

The microcup was made of a non-bioerodible thermoplastic polyetheretherketone. The microcups were made by injection molding using a Battenfeld Microsystem 50 system, and were shaped as a thin, shallow saucer. The microcups were prepared by Rapiwerks, LLC.

### Example 8

### Subconjunctival Placement of a Rapamycin-Containing Solid Drug Delivery System

Approximately 0.5 mg of the solid drug delivery system described in Example 7 were placed between the sclera and the conjunctiva of the eye of New Zealand white rabbits as described in Example 2. The therapeutic agent portion of the solid drug delivery system was placed against the sclera, where it adheres due to ocular moisture. The microcup portion of the solid drug delivery system was oriented towards the conjunctiva, to limit diffusion of rapamycin towards the conjunctiva.

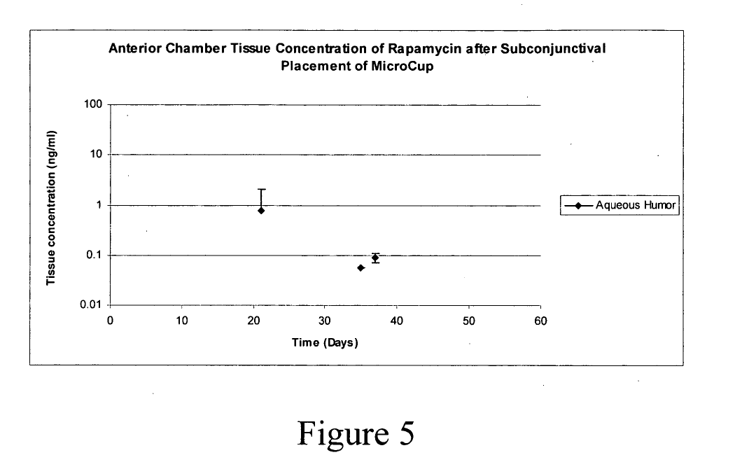

FIG. 5 depicts the level of rapamycin present in the aqueous humor (ng/ml) at 21, 35, and 37 days after placement of the solid drug delivery system.

About 50 μl to about 100 μl were removed from the eye of a rabbit by tuberculin syringe with a 29-30 gauage needle while the rabbit was living. The amount of aqueous humor removed determined, and the sample was frozen and later analyzed. The analysis was by liquid chromatography mass spectroscopy. All timepoints represent the average of two eyes of one rabbit.

The average concentration of the aqueous humor was calculated by dividing the mass of rapamycin measured by the volume of aqueous humor analyzed. The sample did not include the solid drug delivery system; thus, this measurement indicated the level of rapamycin delivered to the aqueous humor via the solid drug delivery system.

The day 21 sample was an average of each of two eyes of each of two rabbits (four eyes total); the day 35 sample was a single rabbit eye; the day 37 sample was an average of each of two eyes. The average level of rapamycin in the aqueous humor at 21, 35, and 37 days after subconjunctival placement of the solid drug delivery system was about 0.76, 0.056, and 0.09 ng/ml, respectively.

### Example 9

A solid drug delivery system comprising rapamycin was prepared with the following components, where the percent is the weight of the component per weight of the total: 44.62% rapamycin (obtained from LC laboratories (Woburn, Mass.), and Chunghwa Chemical Synthesis & BioTech. Co, Ltd (Taiwan)), 39.77% PVP K90 (obtained from BASF), 9.93% Eudragit RL1000 (obtained from Rohm Pharma Polymers), and 5.68% PEG 400 (obtained from DOW Chemical). This solid drug delivery system was prepared as in Example 1.

The solid drug delivery system was stored at 5° C., and had the stability shown in Table 2, below.

Stability was measured via standard HPLC. Three samples were analyzed at each timepoint.

TABLE 1

| Time | Percent drop from the |
|---|---|
| (months) | formula strength |
| 1 | 0% |
| 2 | 0% |
| 3 | 1.80% |
| 6 | 1.90% |
| 12 | 7.60% |

### Example 10

### Solid Drug Delivery Systems

Nonlimiting examples and variations of solid drug delivery systems were prepared, and are listed in Table 2.

All references cited herein, including patents, patent applications, and publications, are hereby incorporated by reference in their entireties, whether previously specifically incorporated or not.

TABLE 2
SOLID DRUG DELIVERY SYSTEMS
Composition (mg)
%, w/w

Rapa = 0 mg (0%)
PEG 400 = 50%
PVP K90 = 40%
Na CMC = 5%
Mannitol = 5%
Rapa = 0%
PEG 400 = 40%
PVP K90 = 50%
Na CMC medium visc. = 10%
Rapa = 6.8 mg (11%)
PVP K90 = 30.5 mg (50%)
Na CMC = 9 mg (14.5%)
PEG 400 = 15 mg (24.5%)
Rapa = 20.7 mg (10.2%)
PVP K90 = 182.2 mg (89.8%)
Rapa = 0 mg (0%)
PVP K90 = 0.801 g (80%)
Eudragit RL100 = 0.1024 g (10%
PEG 400 = 0.9982 (10%)
Rapa = 0 mg (0%)
PVP K90 = 0.902 g (90%)
Eudragit RL100 = 0.1034 g (10%)
Rapa = 0 mg (0%)
PVP K90 = 0.806 g (80%)
Eudragit RL100 = 0.1964 g (20%)
Rapa = 0 mg (0%)
PVP K90 = 0.700 g (70%)
Eudragit RL100 = 0.2978 g (30%)
Rapa = 0 mg (0%)
PVP K90 = 0.598 g (60%)
Eudragit RL100 = 0.398 g (40%)
Rapa = 0 mg (0%)
PVP K90 = 0.502 g (50%)
Eudragit RL100 = 0.513 g (50%)
Rapa = 20 mg (10%)
PVP K90 = 0.090 g (45%)
Eudragit RL100 = 0.090 g (45%)
Rapa = 20 mg (10%)
PVP K90 = 0.080 g (40%)
Eudragit RL100 = 0.080 g (40%)
PEG 400 = 20 mg (10%)
Rapa = 128 mg (47.7%)
PVP K90 = 62.4 mg (23.25%)
Eudragit RL100 = 62.4 mg
(23.25%)
PEG 400 = 15.6 mg (5.8%)
Rapa = 0 mg (0%)
PVP K90 = 45%
PVAP = 45%
PEG 400 = 10%
Rapa = 0.5028 g (50%)
PVP K90 = 0.3013 g (30%)
Eudragit RL100 = 0.0983 g (10%)
PEG 400 = 0.1074 g (10%)
Rapa = 0.4525 g (45.13%)
PVP K90 = 0.4049 g (40.03%)
Eudragit RL100 = 0.0981 g (9.7%)
PEG 400 = 0.0521 g (5.14%)
Rapa = 0.9043 g (44.62%)
PVP K90 = 0.806 g (39.77%)
Eudragit RL100 = 0.2012 g
(9.93%)
PEG 400 = 0.1151 g (5.68%)
PVP (Kollidon K90) = 67.6 mg
(21.78%)
PEG 400 = 76.1 mg (24.56%)
Rapa = 59.9 mg (19.33%)
EtOH = 106.3 mg (34.33%)
Rapa = 40.7 mg (2.02%)
EtOH = 89.2 mg (4.43%)
PEG 1450 = 1885.4 mg (93.55%)
Rapa = 40.2 mg (1.70%)
EtOH = 84.8 mg (4.23%)
PEG 1450 = 1504.48 mg (75.01%)
PEG 600 = 376.12 mg (19.06%)
Rapa = 41.0 mg (2.02%)
EtOH = 88.1 mg (4.39%)
PEG 1450 = 1139.58 mg (56.15%)
PEG 600 = 759.72 mg (37.44%)
Rapa = 40.0 mg (1.98%)
EtOH = 84.2 mg (4.18%)
PEG 1450 = 945.50 mg (46.92%)
PEG 600 = 945.50 mg (46.92%)
Rapa = 41.2 mg (2.05%)
EtOH = 85.1 mg (4.23%)
PEG 1450 = 754.60 mg (37.49%)
PEG 600 = 1131.90 mg (56.24%)

What is claimed is:

1. A solid drug delivery system comprising rapamycin, and wherein the solid drug delivery system when placed between the sclera and conjunctiva of a rabbit eye delivers an amount of rapamycin with a delivery profile selected from the group consisting of
(a) the rapamycin is delivered in an amount sufficient to achieve, for a period of time of at least 90 days following administration of the solid drug delivery system, an average concentration of rapamycin in the vitreous of the rabbit eye of at least 0.01 ng/ml; and
(b) the rapamycin is delivered in an amount sufficient to achieve, for a period of time of at least 90 days following administration of the solid drug delivery system, an average concentration of rapamycin in the retina choroid of the rabbit eye of at least 1 pg/mg.

2. The solid drug delivery system of claim 1, wherein the solid drug delivery system when placed between the sclera and conjunctiva of a rabbit eye delivers an amount of rapamycin with a delivery profile selected from the group consisting of
(a) the rapamycin is delivered in an amount sufficient to achieve, for a period of time of at least 90 days following administration of the solid drug delivery system, an average concentration of rapamycin in the vitreous of the rabbit eye of at least 0.1 ng/ml; and
(b) the rapamycin is delivered in an amount sufficient to achieve, for a period of time of at least 90 days following administration of the solid drug delivery system, an average concentration of rapamycin in the retina choroid of the rabbit eye of at least 10 pg/mg.

3. The solid drug delivery system of claim 1, wherein the solid drug delivery system when placed between the sclera and conjunctiva of a rabbit eye delivers an amount of rapamycin with a delivery profile selected from the group consisting of
(a) the rapamycin is delivered in an amount sufficient to achieve, for a period of time of at least 90 days following administration of the solid drug delivery system, an average concentration of rapamycin in the vitreous of the rabbit eye of at least 1 ng/ml; and
(b) the rapamycin is delivered in an amount sufficient to achieve, for a period of time of at least 90 days following administration of the solid drug delivery system, an average concentration of rapamycin in the retina choroid of the rabbit eye of at least 100 pg/mg.

4. A solid drug delivery system comprising a therapeutic agent, wherein the solid drug delivery system when placed between the sclera and conjunctiva of a rabbit eye delivers an amount of the therapeutic agent with a delivery profile selected from the group consisting of
(a) the the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.01 ng/ml; and
(b) the the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 1 pg/mg.

5. The solid drug delivery system of claim 4, wherein the therapeutic agent is a limus compound.

6. The solid drug delivery system of claim 4, wherein the therapeutic agent is selected from the group consisting of rapamycin, SDZ-RAD, tacrolimus, everolimus, pimecrolimus, CCI-779, AP23841, ABT-578, cyclophilins, TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, and pharmaceutically acceptable salts and esters thereof.

7. The solid drug delivery system of claim 6, wherein the therapeutic agent is selected from the group consisting of rapamycin, SDZ-RAD, tacrolimus, everolimus, pimecrolimus, CCI-779, AP23841, ABT-578, and pharmaceutically acceptable salts and esters thereof.

8. The solid drug delivery system of either of claims 1 or 7, wherein the solid drug delivery system has a backing portion that is at least partially impermeable to the therapeutic agent.

9. The solid drug delivery system of claim 4, wherein the solid drug delivery system when placed between the sclera and conjunctiva of a rabbit eye delivers an amount of the therapeutic agent with a delivery profile selected from the group consisting of
(a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.1 ng/ml; and
(b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 10 pg/mg.

10. The solid drug delivery system of claim 9, wherein the solid drug delivery system when placed between the sclera and conjunctiva of a rabbit eye delivers an amount of the therapeutic agent with a delivery profile selected from the group consisting of
(a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 1 ng/ml; and
(b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 0.05 pg/mg.

11. The solid drug delivery system of claim 1, wherein the rapamycin is present in an amount between 1% and 60% w/w of the drug delivery system.

12. The solid drug delivery system of claim 1, comprising a polyvinylpyrrolidone in an amount between 15% and 45% w/w of the solid drug delivery system.

13. The solid drug delivery system of claim 1, comprising a polyacrylate in an amount between 5% and 30% w/w of the solid drug delivery system.

14. The solid drug delivery system of claim 1, wherein the rapamycin is present in an amount between 1% and 60% w/w of the drug delivery system, further comprising a polyvinylpyrrolidone in an amount between 15% and 45% w/w of the solid drug delivery system, and a polyacrylate in an amount between 5% and 30% w/w of the solid drug delivery system.

15. The solid drug delivery system of claim 1, wherein the solid drug delivery system contains between 20 μg and 4 mg of rapamycin.

16. The solid drug delivery system of claim 1, wherein the solid drug delivery system contains between 20 μg and 2.5 mg of rapamycin.

17. A method for treating wet age-related macular degeneration in a human subject, the method comprising placing the solid drug delivery system of either of claims 1 or 4 proximal to the eye of a human subject in need of treatment of age related macular degeneration.

18. A method for preventing wet age-related macular degeneration in a human subject, the method comprising placing the solid drug delivery system of either of claims 1 or 4 proximal to the eye of the human subject in need of prevention of age related macular degeneration.

19. The method of claim 17, wherein the eye has a sclera with an outer scleral surface and the solid drug delivery system is placed proximal to the outer scleral surface or within a scleral flap.

20. The method of claim 17, wherein the solid drug delivery system is placed between the sclera and conjunctiva.

21. The method of claim 18, wherein the human subject is identified as being at heightened risk of developing wet age-related macular degeneration in the eye to which the solid drug delivery system is administered.

22. The method of claim 21, wherein the human subject has dry age-related macular degeneration in at least one eye.

23. The method of claim 21, wherein the human subject has wet age-related macular degeneration in one eye and the solid drug delivery system is administered to the eye without wet age-related macular degeneration.

24. A solid drug delivery system comprising a therapeutic agent, a polyvinylpyrrolidone, and a polyacrylate, wherein the therapeutic agent is selected from the group consisting of rapamycin, SDZ-RAD, tacrolimus, everolimus, pimecrolimus, CCI-779, AP23841, ABT-578, cyclophilins, TAFA-93, RAD-001, temsirolimus, AP23573, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, monoester derivatives of rapamycin, diester derivatives of rapamycin, 27-oximes of rapamycin; 42-oxo analogs of rapamycin; bicyclic rapamycins; rapamycin dimers; silyl ethers of rapamycin; rapamycin arylsulfonates, rapamycin sulfamates, monoesters at positions 31 and 42, diesters at positions 31 and 42, 30-demethoxy rapamycin, and pharmaceutically acceptable salts and esters thereof.

25. A solid drug delivery system comprising a limus compound, a polyvinylpyrrolidone, and a polyacrylate.

26. The solid drug delivery system of claim 24, wherein the therapeutic agent is selected from the group consisting of rapamycin, SDZ-RAD, tacrolimus, everolimus, pimecrolimus, CCI-779, AP23841, ABT-578, and pharmaceutically acceptable salts and esters thereof.

27. The solid drug delivery system of claim 26 which further comprises a polyethylene glycol.

28. The solid drug delivery system of claim 26, wherein the solid drug delivery system when placed between the sclera and conjunctiva of a rabbit eye delivers an amount of the therapeutic agent with a delivery profile selected from the group consisting of
(a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.1 ng/ml; and
(b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 0.01 ng/mg.

29. The solid drug delivery system of claim 28, wherein the solid drug delivery system when placed between the sclera and conjunctiva of a rabbit eye delivers an amount of the therapeutic agent with a delivery profile selected from the group consisting of
(a) the therapeutic agent is delivered in an amount sufficient to achieve, for a period of time of at least 90 days following administration of the solid drug delivery system, an average concentration of the therapeutic agent in the vitreous of the rabbit eye equivalent to a rapamycin concentration of at least 0.5 ng/ml; and
(b) the therapeutic agent is delivered in an amount sufficient to achieve, for a period of time of at least 90 days following administration of the solid drug delivery system, an average concentration of the therapeutic agent in the retina choroid of the rabbit eye equivalent to a rapamycin concentration of at least 0.05 ng/mg.

30. The solid drug delivery system of claim 24, wherein the therapeutic agent is present in an amount between 1% and 60% w/w of the drug delivery system.

31. The solid drug delivery system of claim 24, wherein the polyvinylpyrrolidone is present in an amount between 15% and 45% w/w of the solid drug delivery system.

32. The solid drug delivery system of claim 24, wherein the polyacrylate is present in an amount between 5% and 30% w/w of the solid drug delivery system.

33. The solid drug delivery system of claim 24, wherein the polyacrylate is a polymethacrylate.

34. The solid drug delivery system of claim 24, wherein the therapeutic agent is present in an amount between 1% and 60% w/w of the drug delivery system, the polyvinylpyrrolidone is present in an amount between 15% and 45% w/w of the solid drug delivery system, and the polyacrylate is present in an amount between 5% and 30% w/w of the solid drug delivery system.

35. The solid drug delivery system of claim 26 which comprises a backing portion at least partially impermeable to the therapeutic agent.

36. A method of treating an ocular condition in a subject requiring placement of an ocular device, comprising administering a formulation comprising an anti-proliferative agent proximal to the site selected for placement of the ocular device.

37. The method of claim 36, wherein the formulation is administered prior to, contemporaneous with, or subsequent to placement of the ocular device.

38. The method of claim 36, wherein the anti-proliferative agent is a limus compound, or a pharmaceutically acceptable salt or ester thereof.

39. The method of claim 38, wherein the limus compound is rapamycin.

40. The method of claim 36, wherein the ocular device is a glaucoma drainage device.

41. The method of claim 39, wherein the ocular device comprises a shunt, stent, tube, membrane, valve, or combination of one or more thereof.

42. The method of claim 36, wherein the method reduces cellular proliferation proximal to the ocular device.

43. The method of claim 36, wherein the formulation is a solution, suspension, emulsion, self-emulsifying formulation, in situ gelling formulation, or a solid drug delivery system.

44. The method of claim 36, wherein the formulation delivers an amount of the antiproliferative agent effective to reduce cellular proliferation proximal to the ocular device for a period of at least about 30 days.

45. The method of claim 44, wherein the formulation delivers an amount of the therapeutic agent effective to reduce cellular proliferation proximal to the ocular device for a period of at least about 60 days.

46. The method of claim 45, wherein the formulation delivers an amount of the therapeutic agent effective to reduce cellular proliferation proximal to the ocular device for a period of at least about 90 days.

47. The method of claim 36, wherein the antiproliferative agent is rapamycin and the ocular device is a glaucoma drainage device.
